(19) United States
(12) Patent Application Publication
(10) Pub. No.: US 2010/0022676 A1
(43) Pub. Date: Jan. 28, 2010

(54) PHOTOCURABLE COMPOSITIONS COMPRISING A PHOTOINITIATOR OF THE PHENYLGLYOXYLATE TYPE

(76) Inventors: Jonathan Rogers, White Plains, NY (US); Johannes Benkhoff, Basel (CH); Karin Powell, Lorrach (DE); Tunja Jung, Rheinfelden-Herten (DE); Kurt Dietliker, Allschwill (CH); Pascal Hayoz, Hofstetten (CH); Jean-Luc Birbaum, Binningen (CH); Thomas Vogel, Haltingen (DE); Rinaldo Hüsler, Basel (CH)

Correspondence Address: JoAnn Villamizar;Ciba Corporation/Patent Department, 540 White Plains Road, P.O. Box 2005, Tarrytown, NY, 10591, US

(21) Appl. No.: 12/442,779

(22) PCT Filed: Sep. 24, 2007
(86) PCT No.: PCT/EP07/60093

§ 371 Date: Mar. 25, 2009

(30) Foreign Application Priority Data
Oct. 3, 2006 (EP) 06121655.2

(51) Int. Cl. C08F 2/46; C08J 7/18; C07C 69/67

(52) U.S. Cl. 522/20; 522/33; 427/514; 560/51

(57) ABSTRACT

The invention provides a uv-curable colored composition, comprising (a) at least one selected ethylenically unsaturated photopolymerizable compound; (b) at least one selected curing agent; and (c) at least one selected colorant.

The invention pertains to novel photoinitiators and pigmented photocurable compositions as well as to a process for curing the same.

Current UV-curing technology is limited in the ability to cure pigmented colored films with sufficient hiding power. This problem for example particularly remains for yellow, orange, red green and black especially if applied in bright colors. The main reason for this failure is the pigments' intrinsic light absorption properties within the UV-range and short-wavelength visual light, preventing the light to penetrate into the film. Therefore not enough radicals are formed from the photoinitiator to fully cross-link the entire film. Such films show a glossy and dry surface, as light is sufficient for surface curing, however close to the substrate crosslinking is reduced or the coating remains liquid or tacky. Poor through-curing leads to failures of film properties such as reduced adhesion, reduced mechanical and chemical resistance and protection of the substrate. Only white and pale shades opaque films can currently be cured using bisacylphosphine oxide-type photoinitiators. Bright colors so far can only be cured in thin, mostly non-opaque, films. To achieve the cure of a fully opaque film with bright colors, it is often necessary to apply and cure several individual layers, which limits the advantages of the UV-curing technology, for example with respect to energy and space savings.

Phenylglyoxylate type photoinitiators have now been identified to increase the curability, i.e. through-curing, of bright colored UV-curable formulations. Phenylglyoxylic acid esters are known as photoinitiators e.g. from U.S. Pat. No. 4,475,999, U.S. Pat. No. 4,038,164, U.S. Pat. No. 6,048,660, WO 00/56822 and EP 965621, but so far have never been taught to be suitable for curing colored, in particular for yellow, orange, red, green and black, UV-curable formulations. Surprisingly, those photoinitiators show no long wavelength absorption, as do the current state of the art bisacylphosphine oxide photoinitiator types. Phenylglyoxylates show significantly improved curability of bright colors, higher film-thicknesses and higher colorant-loaded compositions can be cured, so that opaque UV-curable films in a single application-curing step are achieved.

Subject of the invention therefore is a photocurable composition, comprising

(a) at least one ethylenically unsaturated photopolymerizable compound;
(b) at least one curing agent; and
(c) at least one colorant;
characterized in that
the ethylenically unsaturated photopolymerizable compound (a) is a polyester acrylate;

the curing agent (b) is a photoinitiator compound of the phenylglyoxylate type; and the colorant (c) is a uv- and short-vis-absorbing non-white colorant.

Compounds of the phenylglyoxylate type correspond to phenylglyoxalic acid, its esters, thioesters, amides and salts and corresponding derivatives, in particular to derivatives with alkoxy-, (optionally substituted)phenoxy-, alkylthio- or (optionally substituted)phenylthio-substituents.

In particular the curing agent of the phenylglyoxylate type is a compound of the formula I or Ia,

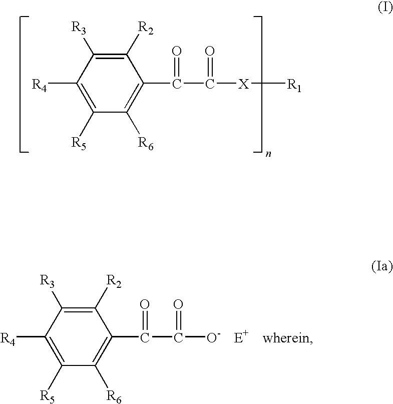

n is 1 or 2;

### X is O, S or NR₁₂;

R₁, if n is 1, is hydrogen; C₁-C₂₀alkyl optionally substituted by OR₇ and/or phenyl; C₂-C₂₀alkyl interrupted by one or more O and optionally substituted by OR₇ and/or phenyl; phenyl optionally substituted by C₁-C₁₂alkyl, cyclopentyl, cyclohexyl, OR₇, SR₇ and/or NR₈R₉; C₃-C₁₂cycloalkyl or C₂-C₁₂alkenyl;
R₁, if n is 2, is C₁-C₂₀alkylene optionally substituted by OR₇ and/or phenyl; C₂-C₂₀alkylene interrupted by one or more O and optionally substituted by OR₇ and/or phenyl; phenylene optionally substituted by C₁-C₁₂alkyl, cyclopentyl, cyclohexyl, OR₇, SR₇ and/or NR₈R₉; C₃-C₁₂cycloalkylene or C₂-C₁₂alkenylene or is one of the groups

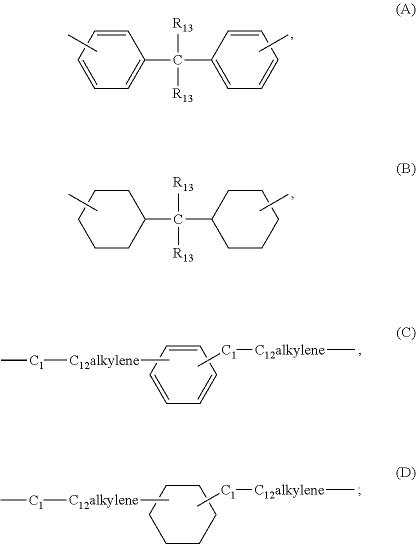

R₂, R₃, R₄, R₅, and R₆ independently of each other are hydrogen; C₁-C₂₀alkyl which optionally is substituted by phenyl, OR₇, SR₇ and/or NR₈R₉; C₂-C₂₀alkyl which is interrupted by one or more O and optionally is substituted by phenyl, OR₇, SR₇ and/or NR₈R₉; C₃-C₁₂cycloalkyl;
C₂-C₁₂alkenyl; phenyl which optionally is substituted by one or more C₁-C₁₂alkyl, OR₇, SR₇ and/or NR₈R₉; or are OR₇, SR₇, or NR₈R₉;
R₇ is hydrogen, C₁-C₂₀alkyl, optionally substituted by OH, OR₁₀ and/or phenyl; C₂-C₂₀alkyl interrupted by one or more O and optionally substituted by OH, OR₁₀ and/or phenyl; phenyl, optionally substituted by one or more C₁-C₁₂alkyl;
R₈ and R₉ independently of each other are hydrogen, C₁-C₂₀alkyl, optionally substituted by OH, OR₁₀ and/or phenyl; C₂-C₂₀alkyl interrupted by one or more O and optionally substituted by OH, OR₁₀ and/or phenyl; phenyl, optionally substituted by one or more C₁-C₁₂alkyl; COR₁₁;
or R₈ and R₉ together with the N-atom to which they are attached form a 5-, 6- or 7-membered ring which optionally is interrupted by O or by NR₁₂;
and wherein R₇, R₈ or R₈ as OR₇, SR₇ or NR₈R₉ with further substituents at the phenyl ring or with a C-atom of the phenyl ring form a 5- or 6-membered ring;
R₁₀ is C₁-C₂₀alkyl;
R₁₁ is C₁-C₂₀alkyl or OR₁₀;
R₁₂ is hydrogen; C₁-C₂₀alkyl, optionally substituted by phenyl, OH and/or OR₁₀; C₂-C₂₀alkyl interrupted by one or more O and optionally substituted by phenyl, OH and/or OR₁₀; C₃-C₁₂cycloalkyl; phenyl, optionally substituted by one or more C₁-C₁₂alkyl, OR₇, SR₇ and/or NR₈R₉;
R₁₃ is hydrogen or C₁-C₂₀alkyl;
E is a cation.

C₁-C₂₀alkyl is linear or branched and is, for example, C₁-C₁₈-, C₁-C₁₄-, C₁-C₁₂-, C₁-C₈-, C₁-C₆- or C₁-C₄alkyl. Examples are methyl, ethyl, propyl, isopropyl, n-butyl, sec-butyl, isobutyl, tert-butyl, pentyl, hexyl, heptyl, 2,4,4-trimethylpentyl, 2-ethylhexyl, octyl, nonyl, decyl, dodecyl, tetradecyl, pentadecyl, hexadecyl, octadecyl and icosyl.

Branched C₃-C₂₀alkyl is for example branched C₃-C₁₈-, C₃-C₁₄-, C₃-C₁₂-, C₃-C₈-, C₃-C₆- or C₃-C₄alkyl, such as isopropyl, sec-butyl, isobutyl, tert-butyl, 2,2-dimethylpropyl, 1-(1-methylethyl)-2-methyl-propyl, 2,4,4-trimethylpentyl, 2-ethylhexyl etc., in particular isopropyl, isobutyl, 2,2-dimethylpropyl or 1-(1-methylethyl)-2-methyl-propyl.

C₁-C₁₈alkyl, C₁-C₁₄alkyl, C₁-C₁₂alkyl, C₁-C₈alkyl, C₁-C₆alkyl and C₁-C₄alkyl have the same meanings as given above for C₁-C₂₀alkyl up to the corresponding number of C-atoms.

C₂-C₂₀alkyl interrupted by one or more O is for example interrupted 1-9, 1-7 or once or twice by O. In case the groups are interrupted by more than one O, said O-atoms are separated from one another by at least one methylene group, i.e. the O-atoms are non-consecutive. Examples are the following structural units —CH₂—O—CH₃, —CH₂CH₂O—CH₂CH₃, —[CH₂CH₂O]_{y}—CH₃, with y=1-9, —(CH₂CH₂O)₇CH₂CH₃, —CH₂—CH(CH₃)—O—CH₂—CH₂CH₃, or —CH₂—CH(CH₃)—O—CH₂CH₃.

C₃-C₁₂cycloalkyl is for example cyclopropyl, cyclopentyl, cyclohexyl, cyclooctyl, cyclododecyl, especially cyclopentyl and cyclohexyl, preferably cyclohexyl. C₃-C₁₂cycloalkyl in the context of the present application is to be understood as alkyl which at least comprises one ring. For example methyl-cyclopentyl, methyl- or dimethylcyclohexyl,

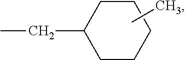

as well as bridged or fused ring systems, e.g.

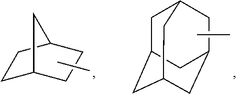

etc. are also meant to be covered by the term.

C₅-C₈cycloalkyl, which is unsubstituted or substituted by linear or branched C₁-C₂₀alkyl is for example cyclopentyl, cyclohexyl, cyclooctyl, cyclo-dodecyl, especially cyclopentyl and cyclohexyl, methylcyclopentyl, methylcyclohexyl, ethylcyclopentyl, ethylcyclohexyl, propylcyclopentyl, propylcyclohexyl, isopropylcyclopentyl, isopropylcyclohexyl, t-butylcyclopentyl, t-butylcyclohexyl, etc.

C₂-C₁₂alkenyl is mono or polyunsaturated, linear or branched and is for example C₂-C₈-, C₂-C₆- or C₂-C₄alkenyl. Examples are allyl, methallyl, vinyl, 1,1-dimethylallyl, 1-butenyl, 3-butenyl, 2-butenyl, 1,3-pentadienyl, 5-hexenyl or 7-octenyl, especially allyl or vinyl.

Substituted phenyl is for example substituted one to five times, e.g. once, twice or three times, in particular once or twice at the phenyl ring.

C₁-C₂₀alkylene is linear or branched alkylene, for example methylene, ethylene, propylene, 1-methylethylene 1,1-dimethylethylene, butylene, 1-methylpropylene, 2-methyl-propylene, pentylene, hexylene, heptylene, octylene, nonylene, decylene, dodecylene, tetradecylene, hexadecylene or octadecylene. In particular, X is C₁-C₁₂alkylene, for example ethylene, decylene,

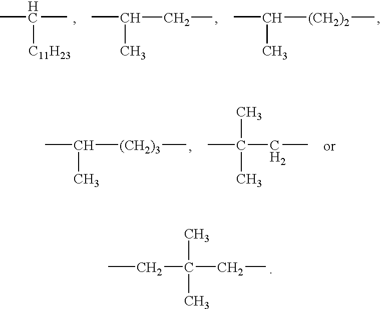

C₂-C₂₀alkylene interrupted by one or more O is, for example, interrupted 1-9 times, for example 1-7 times or once or twice by O. This produces structural units such as, for example, —CH₂—O—CH₂—, —CH₂CH₂—O—CH₂CH₂—, —[CH₂CH₂O]_{y}—, —[CH₂CH₂O]_{y}—CH₂—, where y=1-9, —(CH₂CH₂O)₇CH₂CH₂—, —CH₂—CH(CH₃)—O—CH₂—CH(CH₃)— or —CH₂—CH(CH₃)—O—CH₂—CH₂CH₂—. The interrupting atoms are non-successive.

Branched C₃-C₂₀alkylene is for example 1-methylethylene, 1,1-dimethylethylene, 1,2-dimethylethylene, 1-methylpropylene, 2-methyl-propylene, etc., in particular 1,2-dimethylethylene.

C₃-C₁₂cycloalkylene is, for example, cyclopropylene, cyclopentylene, cyclohexylene, cyclooctylene, cyclododecylene, especially cyclopentylene and cyclohexylene, preferably cyclohexylene. C₃-C₁₂Cycloalkylene is also, however, for example, structural units such as

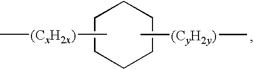

in which x and y independently of one another are 0-6 and the sum of x+y is ≦6, or

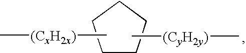

in which x and y independently of one another are 0-7 and the sum of x+y is ≦7.

C₂-C₁₂alkenylene is mono- or polyunsaturated and is, for example, ethenylene, 1-propenylene, 1-butenylene, 3-butenylene, 2-butenylene, 1,3-pentadienylene, 5-hexenylene or 7-octenylene.

If R₇, R₈ or R₉ as OR₇, SR₇ or NR₈R₉ with further substituents at the phenyl ring or with a C-atom of the phenyl ring form a 5- or 6-membered ring, for example the following structures are covered

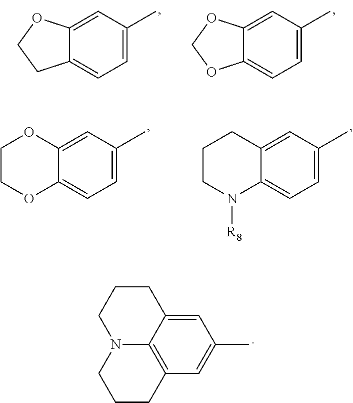

If R₈ and R₈, together with the nitrogen atom to which they are linked, form a 5-, 6- or 7-membered ring which may be interrupted by —O— or by —NR₁₁—, saturated or unsaturated rings are formed, for example aziridine, pyrrole, pyrrolidine, oxazol, pyridine, 1,3-diazine, 1,2-diazine, piperidine or morpholine.

E is a radical able to form positive ions and accordingly is a radical suitable as a counterion E⁺ to the negative phenylglyoxylate ion of the formula Ia. Suitable ions are anorganic or organic cations.

Examples of these E are alkali metals, such as Li, Na, K, or Cs, especially lithium or sodium, alkaline earth metals, such as for example Mg, Ca, Zn, Cu; metal cations in the oxidation state 3+, such as for example Al, metal cations in the oxidation state 4+, such as for example Sn or Ti (it is evident that in case of a counter ion with more than 1 positive valence the respective number of phenylglyoxylate anions is present); “onium” cations, for example quaternary ammonium compounds, ammonium, tetra-alkylammonium, tri-alkyl-aryl-ammonium, di-alkyl-di-aryl-ammonium, tri-aryl-alkyl-ammonium, tetra-aryl-ammonium, tetra-alkylphosphonium, tri-alkyl-aryl-phosphonium, di-alkyl-di-aryl-phosphonium, tri-aryl-alkyl-phosphonium, tetra-aryl-phosphonium, especially ammonium or tetraalkylammonium.

Examples of tetraalkylammonium are, in particular, tetramethylammonium and tetrabutylammonium, although trisalkylammonium ions, for example trimethylammonium, are also suitable. Suitable phosphonium and ammonium counterions are those of the formulae ⁺PR_{w}RₓR_{y}R_{z}, and ⁺NR_{w}RₓR_{y}R_{z}, where R_{w}, Rₓ, R_{y}, R_{z}, independently of one another are hydrogen, unsubstituted or substituted alkyl, cycloalkyl, alkenyl, phenyl or arylalkyl. Substituents for these alkyl, cycloalkyl, alkenyl, phenyl or arylalkyl radicals are, for example, halide, hydroxyl, heterocycloalkyl (e.g. epoxy, aziridyl, oxetanyl, furanyl, pyrrolidinyl, pyrrolyl, thiophenyl, tetrahydrofuranyl, etc.), dialkylamino, amino, carboxyl, alkyl- and arylcarbonyl and aryloxy- and alkoxycarbonyl.

The tetravalent nitrogen may also be part of a 5- or 6-membered ring, in which case this ring may in turn be fused to other ring systems. These systems may also contain additional heteroatoms, for example S, N, O. The tetravalent nitrogen may also be part of a polycyclic ring system, for example azoniapropellane. These systems may also contain further heteroatoms, for example S, N, O.

Also suitable are polyammonium salts and polyphosphonium salts, especially the bis salts, in which it is possible for the same substituents to be present as described above for the “mono” compounds.

Other positive counterions E⁺ to the borate which can be employed are onium ions, for example iodonium or sulfonium ions.

Examples of such counterions to the are radicals of the formula

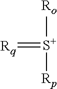

as described in EP 555058 and EP 690074. Also of interest as counterions are

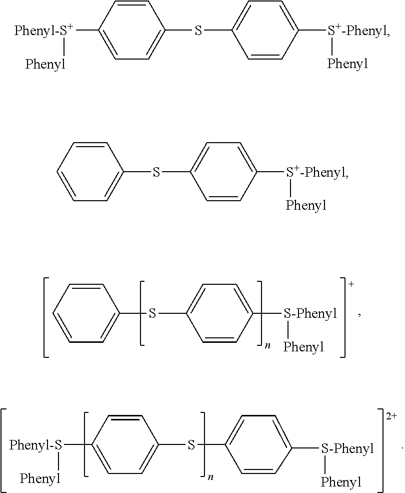

Further interesting as counter ions are iodonium cations, such as for example

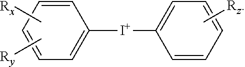

E is for example, a metal cation in the oxidation state+1, N⁺R_{w}RₓR_{y}R_{z} or P⁺R_{w}RₓR_{y}R_{z}, wherein R_{w}, Rₓ, R_{y}, R_{z} independently of one another are hydrogen, C₁-C₂₀alkyl, phenyl; C₁-C₂₀alkyl substituted by OH or phenyl; phenyl substituted by OH or C₁-C₄ alkyl. E is preferably Li⁺, Na⁺, K⁺, Cs⁺, N⁺R_{w}RₓR_{y}R_{z} or P⁺R_{w}RₓR_{y}R_{z}; in particular Li⁺, Na⁺, K⁺, N⁺R_{w}RₓR_{y}R_{z} or P⁺R_{w}RₓR_{y}R_{z}.

Preferred are Na, Li, K, Mg, tetraethylammonium, tetrabutylammonium, tetra(1-hydroxy-3-methyl-but-3-yl)ammonium, tetrabutylphosphonium, triphenylethylphosphonium

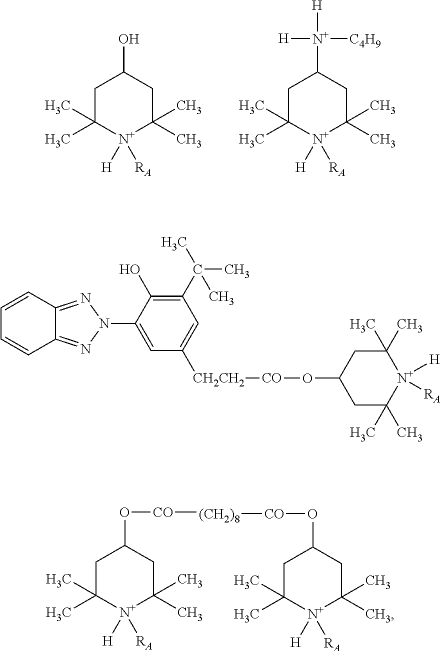

wherein R_{A} is hydrogen or methyl.

Monovalent cations E are preferred.

Phenylglyoxylate compounds are known as photoinitiators. The person skilled in the art is familiar with methods to prepare such compounds and several compounds of this type are commercially available, e.g. IRGACURE® 754, provided by Ciba Specialty Chemicals, or methyl α-oxo benzeneacetate. Examples for the compounds, as well as their preparation are given by J. V. Crivello, K. Dietliker in Photoinitiators for Free Radical Cationic & Anionic Photopolymerization, 2^{nd} ed., Chemistry & Technology of UV&EB Formulation for Coatings, Inks & Paints, Vol. III, 1998, J. Wiley and Sons; by K. Dietliker in A compilation of Photoinitiators Commercially available for UV today, SITA Technology, Edinburgh, 2002; as well as in U.S. Pat. No. 4,038,164, U.S. Pat. No. 1,534,320, U.S. Pat. No. 4,475,999, WO 00/56822, EP 965621, U.S. Pat. No. 6,048,660; WO 06/067061.

Preferred in the composition according to the present invention comprising an ethylenically unsaturated component and a colorant are compounds of the formula I, wherein X is O or NR₁₂, in particular O.

In said compositions R₁, if n is 1 is for example is hydrogen; C₁-C₂₀alkyl optionally substituted by OR₇ and/or phenyl; C₂-C₂₀alkyl interrupted by one or more O and optionally substituted by OR₇ and/or phenyl; phenyl optionally substituted by C₁-C₁₂alkyl, cyclohexyl, OR₇, and/or NR₈R₉; C₅-C₁₂cycloalkyl or C₂-C₁₂alkenyl.

Or R₁, if n is 1, is for example hydrogen; C₁-C₁₂alkyl optionally substituted by OR₇; C₂-C₁₂alkyl interrupted by one or more O and optionally substituted by OR₇; phenyl optionally substituted by C₁-C₄alkyl, OR₇, and/or NR₈R₉; cyclopentyl, cyclohexyl or allyl.

Preferably R₁, if n is 1, is hydrogen; C₁-C₈alkyl optionally substituted by OR₇; C₂-C₁₂alkyl interrupted by one or two O and optionally substituted by OR₇; phenyl optionally substituted by C₁-C₄alkyl, OR₇, and/or NR₈R₉; cyclopentyl, cyclohexyl or allyl.

Especially preferred R₁, if n is 1, is hydrogen; C₁-C₈alkyl optionally substituted by OR₇; C₂-C₁₂alkyl interrupted by one or two O and optionally substituted by OR₇; cyclopentyl, cyclohexyl or allyl and in particular R₁, if n is 1, is hydrogen; C₁-C₆alkyl optionally substituted by methoxy; C₂-C₁₂alkyl interrupted by one or two O and optionally substituted by methoxy; cyclopentyl or, cyclohexyl.

In the compositions of the invention comprising an ethylenically unsaturated component and a colorant in the compounds of the formula I,

R₁, if n is 2, for example denotes C₁-C₂₀alkylene optionally substituted by OR₇ and/or phenyl; C₂-C₂₀alkylene interrupted by one or more O and optionally substituted by OR₇ and/or phenyl; phenylene optionally substituted by C₁-C₁₂alkyl, cyclohexyl, OR₇ and/or NR₈R₉; C₅-C₁₂cycloalkylene or C₂-C₁₂alkenylene or is one of the groups (A), (B), (C) or (D).

Especially R₁, if n is 2, is C₁-C₂₀alkylene; C₂-C₂₀alkylene interrupted by one or more O; phenylene optionally substituted by C₁-C₄alkyl, OR₇ and/or NR₈R₉; C₅-C₁₀cycloalkylene or C₂-C₆alkenylene or is one of the groups (A), (B), (C) or (D).

Preferred R₁, if n is 2, is C₁-C₁₂alkylene; C₂-C₁₂alkylene interrupted by one or more O; phenylene optionally substituted by C₁-C₄alkyl, OR₇ and/or NR₈R₉;

C₅-C₁₀cycloalkylene or C₄-C₆alkenylene, in particular C₁-C₆alkylene; C₂-C₆alkylene interrupted by one or more O.

n preferably is 1.

In the compositions R₂, R₃, R₄, R₅, and R₆ for example independently of each other are C₁-C₁₂alkyl, optionally substituted by phenyl, OR₇, SR₇ and/or NR₈R₉; C₂-C₁₂alkyl interrupted by one or more O and optionally substituted by phenyl, OR₇, SR₇ and/or NR₈R₉; C₅-C₁₂cycloalkyl; C₂-C₁₂alkenyl; phenyl, optionally substituted by one or more C₁-C₁₂alkyl, OR₇, SR₇ and/or NR₈R₉; or are hydrogen, OR₇, SR₇, or NR₈R₉.

In particular R₂ and R₆ are hydrogen; and R₃, R₄ and R₅, independently of each other are C₁-C₄alkyl, optionally substituted by phenyl, OR₇ and/or NR₈R₉; C₂-C₆alkyl interrupted by one or two O; cyclohexyl; allyl; phenyl, optionally substituted by one or two C₁-C₄alkyl, OR₇ and/or NR₈R₉; or are hydrogen, OR₇, SR₇, or NR₈R₉. Preferred are the compositions wherein R₂, R₅, and R₆ are hydrogen; and R₃ and R₄ independently of each other are C₁-C₄alkyl, OR₇, SR₇, or NR₈R₉; and especially such, wherein R₂, R₃, R₅, and R₆ are hydrogen; and R₄ OR₇ or SR₇, in particular SR₇.

R₇ is for example hydrogen, C₁-C₁₂alkyl, optionally substituted by OH, OR₁₀ and/or phenyl; C₂-C₁₂alkyl interrupted by one or more O and optionally substituted by OH, OR₁₀ and/or phenyl; phenyl, optionally substituted by one or more C₁-C₆alkyl. Preferably R₇ is hydrogen, C₁-C₄alkyl; C₂-C₆alkyl interrupted by one or two O and optionally substituted by OH and/or OR₁₀; phenyl, optionally substituted by one or two C₁-C₄alkyl. In particular preferred R₇ is methyl or phenyl.

R₈ and R₉ for example independently of each other are hydrogen, C₁-C₁₂alkyl, optionally substituted by OH, OR₁₀ and/or phenyl; C₂-C₁₂alkyl interrupted by one or more O and optionally substituted by OH, OR₁₀ and/or phenyl; phenyl, optionally substituted by one or more C₁-C₁₂alkyl; COR₁₁; or R₈ and R₉ together with the N-atom to which they are attached form a 5-, 6- or 7-membered ring which optionally is interrupted by O or by NR₁₂; or R₇, R₈ or R₉ as OR₇, SR₇ or NR₈R₉ with further substituents at the phenyl ring or with a C-atom of the phenyl ring form a 5- or 6-membered ring.

R₈ and R₉ for example preferably independently of each other are hydrogen, C₁-C₄alkyl, optionally substituted by OH, OR₁₀ and/or phenyl; C2C₆alkyl interrupted by one or two O and optionally substituted by OH and/or OR₁₀; phenyl, optionally substituted by one or two C₁-C₄alkyl; COR₁₁; or R₈ and R₉ together with the N-atom to which they are attached form a 6-membered ring which optionally is interrupted by O or by NR₁₂; and wherein R₇, R₈ or R₉ as OR₇, SR₇ or NR₈R₉ with further substituents at the phenyl ring or with a C-atom of the phenyl ring form a 5- or 6-membered ring;

R₈ and R₉ in particular independently of each other are hydrogen, C₁-C₄alkyl; C₂-C₆alkyl interrupted by one or two O and optionally substituted by OH and/or OR₁₀; phenyl, optionally substituted by one or two C₁-C₄alkyl or are COR₁₁; or R₈ and R₉ together with the N-atom to which they are attached form a 6-membered ring which optionally is interrupted by O or by NR₁₂.

Especially R₈ and R₉ independently of each other are hydrogen, C₁-C₄alkyl; C₂-C₆alkyl interrupted by one or two O and optionally substituted by OH and/or OR₁₀; phenyl, optionally substituted by one or two C₁-C₄alkyl or are COR₁₁.

R₁₀ is for example C₁-C₁₂alkyl, preferably C₁-C₄alkyl, in particular methyl or ethyl.

R₁₁ is for example C₁-C₁₂alkyl or OR₁₀; preferably C₁-C₄alkyl or OR₁₀; in particular methyl, ethyl or OR₁₀.

R₁₂ for example is hydrogen; C₁-C₂₀alkyl, optionally substituted by phenyl, OH and/or OR₁₀; C₂-C₂₀alkyl interrupted by one or more O and optionally substituted by phenyl, OH and/or OR₁₀; C₃-C₁₂cycloalkyl; phenyl, optionally substituted by one or more C₁-C₁₂alkyl, OR₇, SR₇ and/or NR₈R₉;

R₁₂ preferably is hydrogen; C₁-C₁₂alkyl, optionally substituted by phenyl, OH and/or OR₁₀; C₂-C₁₂alkyl interrupted by one or more O and optionally substituted by phenyl, OH and/or OR₁₀; C₅-C₁₂cycloalkyl; phenyl, optionally substituted by one or more C₁-C₁₂alkyl, OR₇ and/or NR₈R₉.

R₁₂ in particular is hydrogen; C₁-C₄alkyl, optionally substituted by phenyl, OH and/or OR₁₀; C₂-C₆alkyl interrupted by one or two O and optionally substituted by phenyl, OH and/or OR₁₀; cyclohexyl; phenyl, optionally substituted by one or two C₁-C₄alkyl, OR₇ and/or NR₈R₉.

Especially R₁₂ is hydrogen; C₁-C₄alkyl, optionally substituted by OH and/or OR₁₀; C₂-C₆alkyl interrupted by one or two O and optionally substituted by OH and/or OR₁₀.

Interesting are the compounds in the examples below denoted P-14, P-6, P4, P-7, P-9, P-8, P-13, P-3, P-12, P-2, P-10, P-11, P-16, P-17, P-22, P-15, especially P-14, P-6, P-4, P-7, P-9, P-8, P-13, P-3, P-12, P-2, P-10, P-11, in particular P-14, P-6, P4, P-7.

Subject of the invention further are novel phenylglyoxylate compounds of the formula (Ib)

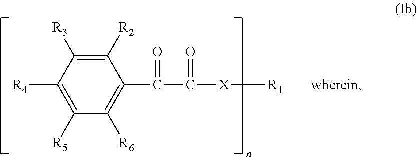

n is 1 or 2;
X is O or S; in particular O;
R₁, if n is 1, is branched C₃-C₂₀alkyl or is C₅-C₈cycloalkyl, which is unsubstituted or substituted by linear or branched C₁-C₂₀alkyl;
R₁, if n is 2, is branched C₃-C₂₀alkylene;
R₂, R₃, R₄, R₅, and R₆ independently of each other are hydrogen; C₁-C₂₀alkyl which optionally is substituted by phenyl, OR₇, SR₇ and/or NR₈R₉; C₂-C₂₀alkyl which is interrupted by one or more O and optionally is substituted by phenyl, OR₇, SR₇ and/or NR₈R₉; C₃-C₁₂cycloalkyl;
C₂-C₁₂alkenyl; phenyl which optionally is substituted by one or more C₁-C₁₂alkyl, OR₇, SR₇ and/or NR₈R₉; or are OR₇, SR₇, or NR₈R₉;
provided that at least one of R₂, R₃, R₄, R₅, and R₆ is SR₇;
R₇ is hydrogen, C₁-C₂₀alkyl, optionally substituted by OH, OR₁₀ and/or phenyl; C₂-C₂₀alkyl interrupted by one or more O and optionally substituted by OH, OR₁₀ and/or phenyl; phenyl, optionally substituted by one or more C₁-C₁₂alkyl;
R₈ and R₉ independently of each other are hydrogen, C₁-C₂₀alkyl, optionally substituted by OH, OR₁₀ and/or phenyl; C₂-C₂₀alkyl interrupted by one or more O and optionally substituted by OH, OR₁₀ and/or phenyl; phenyl, optionally substituted by one or more C₁-C₁₂alkyl; COR₁₁; or R₈ and R₈ together with the N-atom to which they are attached form a 5-, 6- or 7-membered ring which optionally is interrupted by O or by NR₁₂;
and wherein R₇, R₈ or R₈ as OR₇, SR₇ or NR₈R₉ with further substituents at the phenyl ring or with a C-atom of the phenyl ring form a 5- or 6-membered ring;
R₁₀ is C₁-C₂₀alkyl;
R₁₁ is C₁-C₂₀alkyl or OR₁₀; and
R₁₂ is hydrogen; C₁-C₂₀alkyl, optionally substituted by phenyl, OH and/or OR₁₀; C2C₂₀alkyl interrupted by one or more O and optionally substituted by phenyl, OH and/or OR₁₀; C₃-C₁₂cycloalkyl; phenyl, optionally substituted by one or more C₁-C₁₂alkyl, OR₇, SR₇ and/or NR₈R₉.

In particular interesting are those compounds of the formia (Ib), wherein

### R₄ is SR₇;

R₂, R₃, R₅, and R₆ independently of each other are hydrogen; and
R₇ is C₁-C₂₀alkyl.

Further subject of the invention are the compounds

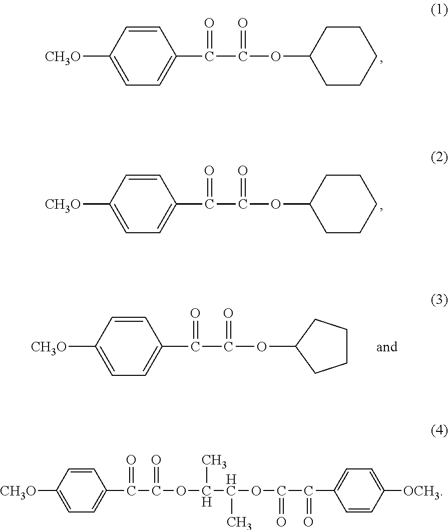

The novel compounds of the formula (Ib) with branched alkyl or cycloalkyl ester groups generally are prepared by transesterification reactions of the corresponding methyl ester compounds.

The preparation of methylester compounds is known and the person skilled in the art is familiar with the appropriate methods. Examples for the preparation of such compounds are given in the literature as cited above and in the preparation examples below.

Preferred are colored, that is non-white, compositions comprising compounds of the formula I or Ib.

Preferably in the compounds of the formula I and Ib, n is 1.

Interesting in particular are compounds of the formula I and Ia, wherein R₂, R₃, R₄, R₅ or R₆ are OR₇, or SR₇, in particular such compounds wherein R₂, R₃, R₄, R₅ or R₆ are OR₇. Preferably R₄ is OR₇.

Further interesting are compounds of the formula I and Ia, wherein R₇ is C₁-C₂₀alkyl, in particular C₁-C₄alkyl, especially methyl, or phenyl. Emphasis has to be laid on compounds of the formula I or Ia, wherein R₄ is OR₇ or SR₇ and R₃ and R₅ are C₁-C₂₀alkyl.

In particular interesting are compounds of the formula I, wherein R₁, if n is 1, is hydrogen, C₁-C₂₀alkyl or C₂-C₂₀alkyl interrupted by one or more O. In particular R₁ is hydrogen, methyl, ethyl or —(CH₂CH₂O)₂—CH₃.

Interesting cations E are in particular metal cations and ammonium ions, especially Li, Na, K, Mg cations and tetraalkylammonium, such as for example tetramethylammonium or tetrabutylammonium.

The compositions of the invention comprise at least one non-white colorant (c). Subject of the invention are photocurable compositions, wherein the colorant (c) is a colored pigment or dye in particular a colored pigment or dye selected from the group consisting of yellow, orange, red, green and black pigments or dyes. Depending on the kind of application organic as well as anorganic pigments are used as colorant. Examples for pigments are inorganic pigments, such as for example titanium dioxide, e.g. of the rutil or anatas type, zinc oxide, such as zink white, zinc sulfide, barium sulfate, aluminium silicate, calcium silicate, carbon black, iron oxides, such as iron oxide yellow, iron oxide red, black iron oxide, iron blue, copper chromite black, chromium oxide greens, chrome green, violet (e.g. manganese violet, cobalt phosphate, CoLiPO₄), chromium yellow, chromium green, lead chromates, lead molybdates, cadmium titanate and pearlescent and metallic pigments, nickel titanium yellow, ultramarine blue, cobalt blue, bismuth vanadate, cadmium yellow or cadmium red; as well as organic pigments, such as monoazo pigments, di-azo pigments, di-azo condensation pigments, as well as metal complexes thereof, polycyclic pigments, such as perylene pigments, anthraquinone pigments, thioindigo pigments, or triphenylmethane pigments, as well as diketopyrrolo-pyrole pigments, isoindolinone pigments, e.g. tetrachlorisoindolinone pigments, isoindoline pigments, dioxazin pigments, benzimidazolone pigments and chinophthalone pigments, quinacridone pigments, dioxazine violet, vat pigments, and phthalocyanine pigments. Examples for suitable pigments include carbon black for a black coating, titanium dioxide for a white coating, diarylide yellow or diazo based pigments for yellow coatings, phthalocyanine blue, and other phthalocyanines for blue coatings, anthraquinone red, naphthole red, monazo based pigments, quinacridone pigments, anthraquinone and perylenes for red coatings, phthalocyanine green and nitroso based pigments for green coatings, monazo and diazo based pigments, quinacridone pigments, anthraquinones and perylenes for orange coatings, and quinacridone violet, basic dye pigments and carbazole dioxazine based pigments for violet coatings. The person skilled in the art is well aware of formulating and combining suitable further pigments if even more colored coatings, such as aqua, brown, gray, pink etc. are needed.

By way of example, examples of organic pigments include Colour Index Pigment Yellow 3, 12, 13, 14, 17, 24, 34, 42, 53, 62, 74, 83, 93, 95, 108, 109, 110, 111, 119, 123, 128, 129, 139, 147, 150, 164, 168, 173, 174, 184, 188, 191, 191:1, 193, 199, Pigment Orange 5, 13, 16, 34, 40, 43, 48, 49, 51, 61, 64, 71, 73, Pigment Red 2, 4, 5, 23, 48:1, 48:2, 48:3, 48:4, 52:2, 53:1, 57, 57:1, 88, 89, 101, 104, 112, 122, 144, 146, 149, 166, 168, 177, 178, 179, 181, 184, 190, 192, 194, 202, 204, 206, 207, 209, 214, 216, 220, 221, 222, 224, 226, 254, 255, 262, 264, 270, 272, Pigment Brown 23, 24, 33, 42, 43, 44, Pigment Violet 19, 23, 29, 31, 37, 42, Pigment Blue 15, 15:1, 15:2, 15:3, 15:4, 15:6, 16, 28, 29, 60, 64, 66, Pigment Green 7, 17, 36, 37, 50, Pigment White 6, Pigment Black 7, 12, 27, 30, 31, 32, Vat Red 74, 3,6-di(3′-cyano-phenyl)-2,5-dihydro-pyrrolo[3,4-c]pyrrole-1,4-dione or 3-phenyl-6-(4′-tert-butyl-phenyl)-2,5-dihydro-pyrrolo[3,4-c]pyrrole-1,4-dione. Further examples of organic pigments can be found in the monograph: Suitable colorants include but are not limited to: 3-dibutylamino-7-dibenzylaminofluoran, 3-diethylamino-6-methylfluoran, 3-dimethylamino-4-methyl-7-anilinofluoran, 3-diethylamino-4-methyl-7-anilinofluoran, 3-diethylamino-4-methyl-7-(2,4-dimethylanilino) fluoran, 3-diethylamino-6-methyl-7-chlorofluoran, 3-diethylamino-6-methyl-7-(3-trifluoromethylanilino)fluoran, 3-diethylamino-6-methyl-7-(2-chloroanilino)fluoran, 3-diethylamino-6-methyl-7-(4-chloroanilino)fluoran, 3-diethylamino-6-methyl-7-(2-fluoroanilino)fluoran, 3-diethylamino-6-methyl-7-(4-n-octylanilino)fluoran, 3-diethylamino-7-(4-n-octylanilino)fluoran, 3-diethylamino-6-methyl-7-(dibenzylamino)fluoran, 3-diethylamino-7-(dibenzylamino)fluoran, 3-diethylamino-6-chloro-7-methylfluoran, 3-diethylamino-7-t-butylfluoran, 3-diethylamino-7-carboxyethylfluoran, 3-diethylamino-6-chloro-7-anilinofluoran, 3-diethylamino-6-methyl-7-(3-methylanilino)fluoran, 3-diethylamino-6-methyl-7-(4-methylanilino)fluoran, 3-diethylamino-6-ethoxyethyl-7-anilinofluoran, 3-diethylamino-7-methylfluoran, 3-diethylamino-6,8-dimethylfluoran, 3-diethylamino-7-chlorofluoran, 3-diethylamino-7-chlorofluoran, 3-diethylamino-7-(3-trifluoromethylanilino) fluoran, 3-diethylamino-7-(2-chloroanilino)fluoran, 3-diethylamino-7-(2-fluoroanilino)fluoran, 3-diethylamino-benzo[a]fluoran, 3-diethylamino-benzo[c]fluoran, 3-dibutylamino-6-methyl fluoran, 3-dibutylamino-6-methyl-7-anilinofluoran, 3-dibutylamino-6-methyl-7-(2,4-dimethylanilino)-fluoran, 3-dibutylamino-4-methyl-7-(2-chloroanilino)fluoran, 3-dibutylamino-6-methyl-7-(4-chloroanilino)fluoran, 3-dibutylamino-6-methyl-7-(2-fluoroanilino)fluoran, 3-dibutylamino-6-methyl-7-(3-trifluoromethylanilino)fluoran, 3-dibutylamino-6-ethoxyethyl-7-anilinofluoran, 3-dibutylamino-6-chloro-anilinofluoran, 3-dibutylamino-6-methyl-7-(4-methylanilino)fluoran, 3-dibutyamino-7-(2-chloroanilino)fluoran, 3-dibutylamino-7-(2-fluoroanilino)fluoran, 3-dipentylamino-6-methyl-7-anilinofluoran, 3-dipentylamino-6-methyl-7-(4-2-chloroanilino)fluoran, 3-dipentylamino-7-(3-trifluoromethylanilino)fluoran, 3-dipentylamino-6-chloro-7-anilinofluoran, 3-dipentylamino-7-(4-chloroanilino)fluoran, 3-pyrrolidino-4-methyl-7-anilinofluoran, 3-piperidino-6-methyl-7-anilinofluoran, 3-(N-methyl-N-propylamino)-6-methyl-7-anilinofluoran, 3-(N-methyl-N-cyclohexylamino)-6-methyl-7-anilinofluoran, 3-(N-ethyl-N-cyclohexylamino)-6-methyl-7-anilinofluoran, 3-(N-ethyl-N-n-hexylamino)-7-anilinofluoran, 3-(N-ethyl-p-toluidino)-amino-6-methyl-7-anilinofluoran, 3-(N-ethyl-p-toluidino)amino-7-methylfluoran, 3-(N-ethyl-N-isoamylamino)-6-methyl-7-anilinofluoran, 3-(N-ethyl-N-isoamylamino)-7-(2-chloroanilino)-fluoran, 3-(N-ethyl-N-isoamylamino)-6-chloro-7-anilinofluoran, 3-(N-ethyl-N-tetrahydrofurfurylamino)-6-methyl-7-anilinofluoran, 3-(N-ethyl-N-isobutylamino)-6-methyl-7-anilinofluoran, 3-(N-butyl-N-isoamylamino)-6-methyl-7-anilinofluoran, 3-(N-isopropyl-N-3-pentylamino)-6-methyl7-anilinofluoran, 3-(N-ethyl-N-ethoxypropylamino)-6-methyl-7-anilinofluoran, 3-cyclohexylamino-6-chlorofluoran, 2-methyl-6-p-(p-dimethylaminophenyl)aminoanilinofluoran, 2-methoxy-6-p-(p-dimethylaminophenyl)aminoanilinofluoran, 2-chloro-3-methyl-6-p-(p-phenylaminophenyl)aminoanilinofluoran, 2-diethylamino-6-p-(p-dimethylaminophenyl)aminoanilinofluoran, 2-phenyl-6-methyl-6-p-(p-phenylaminophenyl)aminoanilinofluoran, 2-benzyl-6-p-(p-phenylaminophenyl)aminoanilinofluoran, 3-methyl-6-p-(p-dimethylaminophenyl)aminoanilinofluoran, 3-diethylamino-6-p-(p-diethylaminophenyl)aminoanilinofluoran, 3-diethylamino-6-p-(p-dibutylaminophenyl)aminoanilinofluoran, 2,4-dimethyl-6-[(4-dimethylamino)-anilino]fluoran.

Suitable are also the colorants as are described by W. Herbst, K. Hunger in “Industrial organic pigments: production, properties, applications” 3rd. completely revised Edition, 2004, Wiley-VCH, Weinheim, ISBN: 3-527-30576-9, which disclosure herby is incorporated by reference.

The colorants may be used as single compounds or in combination with each other or with further color forming compounds.

Preferred are the non-blue pigments, inorganic or organic, as named above.

In the context of the present invention the white pigments named above, such as for example titanium dioxide or zinc oxide, are not considered a colorant (c) as such, but only as “shading” component for brightening the shade of non-white colorants (c). That is, white pigments as named above are not considered a component (c) according to the present invention, unless they are admixed with a non-white colorant.

The pigments may be single chemical compounds or mixtures of a plurality of components, including solid solutions or mixed crystals containing a plurality of chemical compounds. Preference is given to uniformly crystalline pigments as they usually yield greater colour saturation than physical mixtures and mixed phases. If duller shades are nevertheless desired in the final application, this may be achieved by toning down with colorants of different colour in a manner known per se.

Depending on the intended use the pigments are used in amounts customary in the art, for example in an amount of 1 to 60% by weight, or 10 to 30% by weight, based on the whole formulation. Suitable amounts for pigments in an ink coating are for example 1 to 20, 1 to 15, preferably 1 to 10 wt %. The above amounts refer to the total amount of the pigments in the formulation in case that a mixture of different pigments, that is more than one pigment, is employed.

The mean particle size of the pigments usually is about 1 μm or less. The size of commercial pigments can be reduced by milling, if necessary. The pigments for example, can be added to the formulation in the form of a dispersion in order to simplify the mixing with the other ingredients of the formulation. The pigments are, for example dispersed in a low viscosity liquid, e.g. a reactive diluent.

Preferred is the use of organic pigments. In particular preferred in the context of the present invention is the use of colored, i.e. non-white, pigments. Especially preferred are bright shades, i.e. full-shades or ⅓ international standard depth (ISD).

The compositions may also comprise organic dyes of different classes. Examples are azo dyes, methin dyes, anthraquinone dyes or metal complex dyes. Customary concentrations are for example 0.1 to 20%, in particular 1 to 5%, based on the whole formulation.

Suitable colorants are for example selected from the group consisting of spiro-pyrans, spirooxazines, naphthopyrans and lactones.

Examples are fluorans, triphenylmethanes, lactones, benzoxazines, spiropyrans, phthalides; preferably fluorans. Suitable colorants include but are not limited to: 3-dibutylamino-7-dibenzylaminofluoran, 3-diethylamino-6-methylfluoran, 3-dimethylamino-6-methyl-7-anilinofluoran, 3-diethylamino-6-methyl-7-anilinofluoran, 3-diethylamino-6-methyl-7-(2,4-dimethylanilino) fluoran, 3-diethylamino-6-methyl-7-chlorofluoran, 3-diethylamino-6-methyl-7-(3-trifluoromethylanilino)fluoran, 3-diethylamino-6-methyl-7-(2-chloroanilino)fluoran, 3-diethylamino-6-methyl-7-(4-chloroanilino)fluoran, 3-diethylamino-6-methyl-7-(2-fluoroanilino)fluoran, 3-diethylamino-6-methyl-7-(4-n-octylanilino)fluoran, 3-diethylamino-7-(4-n-octylanilino)fluoran, 3-diethylamino-6-methyl-7-(dibenzylamino)fluoran, 3-diethylamino-7-(dibenzylamino)fluoran, 3-diethylamino-4-chloro-7-methylfluoran, 3-diethylamino-7-t-butylfluoran, 3-diethylamino-7-carboxyethylfluoran, 3-diethylamino-6-chloro-7-anilinofluoran, 3-diethylamino-6-methyl-7-(3-methylanilino)fluoran, 3-diethylamino-6-methyl-7-(4-methylanilino)fluoran, 3-diethylamino-6-ethoxyethyl-7-anilinofluoran, 3-diethylamino-7-methylfluoran, 3-diethylamino-6,8-dimethylfluoran, 3-diethylamino-7-chlorofluoran, 3-diethylamino-7-chlorofluoran, 3-diethylamino-7-(3-trifluoromethylanilino) fluoran, 3-diethylamino-7-(2-chloroanilino)fluoran, 3-diethylamino-7-(2-fluoroanilino)fluoran, 3-diethylamino-benzo[a]fluoran, 3-diethylamino-benzo[c]fluoran, 3-dibutylamino-6-methyl fluoran, 3-dibutylamino-6-methyl-7-anilinofluoran, 3-dibutylamino-6-methyl-7-(2,4-dimethylanilino) fluoran, 3-dibutylamino-4-methyl-7-(2-chloroanilino)fluoran, 3-dibutylamino-6-methyl-7-(4-chloroanilino)fluoran, 3-dibutylamino-6-methyl-7-(2-fluoroanilino)fluoran, 3-dibutylamino-6-methyl-7-(3-trifluoromethylanilino)fluoran, 3-dibutylamino-6-ethoxyethyl-7-anilinofluoran, 3-dibutylamino-6-chloro-anilinofluoran, 3-dibutylamino-6-methyl-7-(4-methylanilino)fluoran, 3-dibutyamino-7-(2-chloroanilino)fluoran, 3-dibutylamino-7-(2-fluoroanilino)fluoran, 3-dipentylamino-6-methyl-7-anilinofluoran, 3-dipentylamino-6-methyl-7-(4-2-chloroanilino)fluoran, 3-dipentylamino-7-(3-trifluoromethylanilino)fluoran, 3-dipentylamino-6-chloro-7-anilinofluoran, 3-dipentylamino-7-(4-chloroanilino)fluoran, 3-pyrrolidino-6-methyl-7-anilinofluoran, 3-piperidino-6-methyl-7-anilinofluoran, 3-(N-methyl-N-propylamino)-6-methyl-7-anilinofluoran, 3-(N-methyl-N-cyclohexylamino)-6-methyl-7-anilinofluoran, 3-(N-ethyl-N-cyclohexylamino)-6-methyl-7-anilinofluoran, 3-(N-ethyl-N-n-hexylamino)-7-anilinofluoran, 3-(N-ethyl-p-toluidino)-amino-6-methyl-7-anilinofluoran, 3-(N-ethyl-p-toluidino)amino-7-methylfluoran, 3-(N-ethyl-N-isoamylamino)-6-methyl-7-anilinofluoran, 3-(N-ethyl-N-isoamylamino)-7-(2-chloroanilino)-fluoran, 3-(N-ethyl-N-isoamylamino)-6-chloro-7-anilinofluoran, 3-(N-ethyl-N-tetrahydrofurfurylamino)-6-methyl-7-anilinofluoran, 3-(N-ethyl-N-isobutylamino)-6-methyl-7-anilinofluoran, 3-(N-butyl-N-isoamylamino)-6-methyl-7-anilinofluoran, 3-(N-isopropyl-N-3-pentylamino)-6-methyl7-anilinofluoran, 3-(N-ethyl-N-ethoxypropylamino)-6-methyl-7-anilinofluoran, 3-cyclohexylamino-6-chlorofluoran, 2-methyl-6-p-(p-dimethylaminophenyl)aminoanilinofluoran, 2-methoxy-6-p-(p-dimethylaminophenyl)aminoanilinofluoran, 2-chloro-3-methyl-6-p-(p-phenylaminophenyl)aminoanilinofluoran, 2-diethylamino-6-p-(p-dimethylaminophenyl)aminoanilinofluoran, 2-phenyl-6-methyl-6-p-(p-phenylaminophenyl)aminoanilinofluoran, 2-benzyl-6-p-(p-phenylaminophenyl)aminoanilinofluoran, 3-methyl-6-p-(p-dimethylaminophenyl)aminoanilinofluoran, 3-diethylamino-6-p-(p-diethylaminophenyl)aminoanilinofluoran, 3-diethylamino-6-p-(p-dibutylaminophenyl)aminoanilinofluoran, 2,4-dimethyl-6-[(4-dimethylamino)-anilino]fluoran.

More compounds and their preparation can be found in K. Hunger “Industrial dyes: chemistry, properties, applications”, 2003, Wiley-VCH, Weinheim, ISBN 3-527-30426-6 and in H. Zollinger “Color chemistry: syntheses, properties, and applications of organic dyes and pigments”, 3^{rd} revised edition, 2003, Wiley-VCH, Weinheim, ISBN 3-906390-23-3, both documents hereby are incorporated by reference.

The colorants may be used as single compounds or in combination with each other or with further color forming compounds.

Suitable pigments and dyes are commercially available and known to the person skilled in the art. Specific examples are

Cromophtal Yellow 8GN (P.Y. 128), Irgazin Yellow 2093, Irgalite Yellow GO, Irgacolor Yellow 14247, Irgazin Yellow 2RLT, Irgazin Orange 2038, Irgazin DPP Orange RA (P.O. 73), Irgazin Red 2030, Kronos 2310, Irgalite Res 3RS, Irgazin Green 2180, Special Black 4.

In accordance with the invention, the compounds of the formula I and Ia can be used as photoinitiators for the photopolymerization of compositions comprising ethylenically unsaturated compounds and colorants and the compounds of the formula (Ib), (1), (2), (3) and (4) can be used as photoinitiators for the photopolymerization of compositions comprising ethylenically unsaturated compounds.

Accordingly subject of the invention also is a photopolymerizable composition, comprising

(a) at least one ethylenically unsaturated photopolymerizable compound; and
(b) as photoinitiator at least one compound of the formula (Ib) as defined above or a compound (1), (2), (3) or (4) as defind above.

The composition (comprising compounds of the formula I, Ia, Ib, (1), (2), (3) or (4)) may comprise additionally to the component (b) at least one further photoinitiator (b1), and/or further coinitiators (d) and/or other additives (e).

The unsaturated compounds (a) may include one or more olefinic double bonds. They may be of low (monomeric) or high (oligomeric) molecular mass. Examples of monomers containing a double bond are alkyl or hydroxyalkyl acrylates or methacrylates, for example methyl, ethyl, butyl, 2-ethylhexyl or 2-hydroxyethyl acrylate, isobornyl acrylate, methyl methacrylate or ethyl methacrylate. Interesting also are resins which are modified with silicon or fluor, e.g. silicon acrylates. Other examples are acrylonitrile, acrylamide, methacrylamide, N-substituted (meth)acrylamides, vinyl esters such as vinyl acetate, vinyl ethers such as isobutyl vinyl ether, styrene, alkyl- and halostyrenes, N-vinylpyrrolidone, vinyl chloride or vinylidene chloride.

Examples of monomers containing two or more double bonds are the diacrylates of ethylene glycol, propylene glycol, neopentyl glycol, hexamethylene glycol or of bisphenol A, and 4,4′-bis(2-acryloyloxyethoxy)diphenylpropane, trimethylolpropane triacrylate, pentaerythritol triacrylate or tetraacrylate, vinyl acrylate, divinylbenzene, divinyl succinate, diallyl phthalate, triallyl phosphate, triallyl isocyanurate or tris(2-acryloylethyl)isocyanurate.

Examples of polyunsaturated compounds of relatively high molecular mass (oligomers) are acrylisized epoxy resins, acrylisized polyesters, polyesters containing vinyl ether or epoxy groups, and also polyurethanes and polyethers. Further examples of unsaturated oligomers are unsaturated polyester resins, which are usually prepared from maleic acid, phthalic acid and one or more diols and have molecular weights of from about 500 to 3000. In addition it is also possible to employ vinyl ether monomers and oligomers, and also maleate-terminated oligomers with polyester, polyurethane, polyether, polyvinyl ether and epoxy main chains. Of particular suitability are combinations of oligomers which carry vinyl ether groups and of polymers as described in WO 90/01512. However, copolymers of vinyl ether and maleic acid-functionalized monomers are also suitable. Unsaturated oligomers of this kind can also be referred to as prepolymers.

Particularly suitable examples are esters of ethylenically unsaturated carboxylic acids and polyols or polyepoxides, and polymers having ethylenically unsaturated groups in the chain or in side groups, for example unsaturated polyesters, polyamides and polyurethanes and copolymers thereof, alkyd resins, polybutadiene and butadiene copolymers, polyisoprene and isoprene copolymers, polymers and copolymers containing (meth)acrylic groups in side chains, and also mixtures of one or more such polymers.

Examples of unsaturated carboxylic acids are acrylic acid, methacrylic acid, crotonic acid, itaconic acid, cinnamic acid, and unsaturated fatty acids such as linolenic acid or oleic acid. Acrylic and methacrylic acid are preferred.

Suitable polyols are aromatic and, in particular, aliphatic and cycloaliphatic polyols. Examples of aromatic polyols are hydroquinone, 4,4′-dihydroxydiphenyl, 2,2-di(4-hydroxyphenyl)propane, and also novolaks and resols. Examples of polyepoxides are those based on the abovementioned polyols, especially the aromatic polyols, and epichlorohydrin. Other suitable polyols are polymers and copolymers containing hydroxyl groups in the polymer chain or in side groups, examples being polyvinyl alcohol and copolymers thereof or polyhydroxyalkyl methacrylates or copolymers thereof. Further polyols which are suitable are oligoesters having hydroxyl end groups.

Examples of aliphatic and cycloaliphatic polyols are alkylenediols having preferably 2 to 12 C atoms, such as ethylene glycol, 1,2- or 1,3-propanediol, 1,2-, 1,3- or 1,4-butanediol, pentanediol, hexanediol, octanediol, dodecanediol, diethylene glycol, triethylene gicyol, polyethylene glycols having molecular weights of preferably from 200 to 1500, 1,3-cyclopentanediol, 1,2-, 1,3- or 1,4-cyclohexanediol, 1,4-dihydroxymethylcyclohexane, glycerol, tris(β-hydroxyethyl)amine, trimethylolethane, trimethylolpropane, pentaerythritol, dipentaerythritol and sorbitol.

The polyols may be partially or completely esterified with one carboxylic acid or with different unsaturated carboxylic acids, and in partial esters the free hydroxyl groups may be modified, for example etherfied or esterified with other carboxylic acids.

Examples of esters are:

trimethylolpropane triacrylate, trimethylolethane triacrylate, trimethylolpropane trimethacrylate, trimethylolethane trimethacrylate, tetramethylene glycol dimethacrylate, triethylene glycol dimethacrylate, tetraethylene glycol diacrylate, pentaerythritol diacrylate, pentaerythritol triacrylate, pentaerythdtol tetraacrylate, dipentaerythritol diacrylate, dipentaerythritol triacrylate, dipentaerythritol tetraacrylate, dipentaerythritol pentaacrylate, dipentaerythritol hexaacrylate, tripentaerythritol octaacrylate, pentaerythritol dimethacrylate, pentaerythritol trimethacrylate, dipentaerythritol dimethacrylate, dipentaerythritol tetramethacrylate, tripentaerythritol octamethacrylate, pentaerythritol diitaconate, dipentaerythritol tris-itaconate, dipentaerythritol pentaitaconate, dipentaerythritol hexaitaconate, ethylene glycol diacrylate, 1,3-butanediol diacrylate, 1,3-butanediol dimethacrylate, 1,4-butanediol diitaconate, sorbitol triacrylate, sorbitol tetraacrylate, pentaerythritol-modified triacrylate, sorbitol tetra methacrylate, sorbitol pentaacrylate, sorbitol hexaacrylate, oligoester acrylates and methacrylates, glycerol diacrylate and triacrylate, 1,4-cyclohexane diacrylate, bisacrylates and bismethacrylates of polyethylene glycol with a molecular weight of from 200 to 1500, or mixtures thereof.

Also suitable as components (a) are the amides of identical or different, unsaturated carboxylic acids with aromatic, cycloaliphatic and aliphatic polyamines having preferably 2 to 6, especially 2 to 4, amino groups. Examples of such polyamines are ethylenediamine, 1,2- or 1,3-propylenediamine, 1,2-, 1,3- or 1,4-butylenediamine, 1,5-pentylenediamine, 1,6-hexylenediamine, octylenediamine, dodecylenediamine, 1,4-diaminocyclohexane, isophoronediamine, phenylenediamine, bisphenylenediamine, di-β-aminoethyl ether, diethylenetriamine, triethylenetetramine, di(β-aminoethoxy)- or di(β-aminopropoxy)ethane. Other suitable polyamines are polymers and copolymers, preferably with additional amino groups in the side chain, and oligoamides having amino end groups. Examples of such unsaturated amides are methylenebisacrylamide, 1,6-hexamethylenebisacrylamide, diethylenetriamine-trismethacrylamide, bis(methacrylamidopropoxy)ethane, β-methacrylamidoethyl methacrylate and N[(β-hydroxyethoxy)ethyl]acrylamide.

Suitable unsaturated polyesters and polyamides are derived, for example, from maleic acid and from diols or diamines. Some of the maleic acid can be replaced by other dicarboxylic acids. They can be used together with ethylenically unsaturated comonomers, for example styrene. The polyesters and polyamides may also be derived from dicarboxylic acids and from ethylenically unsaturated diols or diamines, especially from those with relatively long chains of, for example 6 to 20 C atoms. Examples of polyurethanes are those composed of saturated or unsaturated diisocyanates and of unsaturated or, respectively, saturated diols.

Polybutadiene and polyisoprene and copolymers thereof are known. Examples of suitable comonomers are olefins, such as ethylene, propene, butene and hexene, (meth)acrylates, acrylonitrile, styrene or vinyl chloride. Polymers with (meth)acrylate groups in the side chain are likewise known. They may, for example, be reaction products of epoxy resins based on novolaks with (meth)acrylic acid, or may be homo- or copolymers of vinyl alcohol or hydroxyalkyl derivatives thereof which are esterified with (meth)acrylic acid, or may be homo- and copolymers of (meth)acrylates which are esterified with hydroxyalkyl (meth)acrylates.

The photopolymerizable compounds can be used alone or in any desired mixtures. It is preferred to use mixtures of polyol (meth)acrylates.

Binders as well can be added to these novel compositions, and this is particularly expedient when the photopolymerizable compounds are liquid or viscous substances. The quantity of binder may, for example, be 5-95%, preferably 10-90% and especially 40-90%, by weight relative to the overall solids content. The choice of binder is made depending on the field of application and on properties required for this field, such as the capacity for development in aqueous and organic solvent systems, adhesion to substrates and sensitivity to oxygen.

Examples of suitable binders are polymers having a molecular weight of about 5000 to 2000000, preferably 10000 to 1000000. Examples are: homo- and copolymers of acrylates and methacrylates, for example copolymers of methyl methacrylate/ethyl acrylate/methacrylic acid, poly(alkyl methacrylates), poly(alkyl acrylates); cellulose esters and cellulose ethers, such as cellulose acetate, cellulose acetobutyrate, methylcellulose, ethylcellulose; polyvinylbutyral, polyvinylformal, cyclized rubber, polyethers such as polyethylene oxide, polypropylene oxide and polytetrahydrofuran; polystyrene, polycarbonate, polyurethane, chlorinated polyolefins, polyvinyl chloride, vinyl chloride/vinylidene copolymers, copolymers of vinylidene chloride with acrylonitrile, methyl methacrylate and vinyl acetate, polyvinyl acetate, copoly(ethylene/vinyl acetate), polymers such as polycaprolactam and poly-(hexamethylenadipamide), and polyesters such as poly(ethylene glycol terephtalate) and poly(hexamethylene glycol succinate) and polyimides.

The unsaturated compounds can also be used as a mixture with non-photopolymerizable, film-forming components. These may, for example, be physically drying polymers or solutions thereof in organic solvents, for instance nitrocellulose or cellulose acetobutyrate. They may also, however, be chemically and/or thermally curable (heat-curable) resins, examples being polyisocyanates, polyepoxides and melamine resins, as well as polyimide precursors. The use of heat-curable resins at the same time is important for use in systems known as hybrid systems, which in a first stage are photopolymerized and in a second stage are crosslinked by means of thermal aftertreatment.

In the composition according to the invention, comprising compounds of the formula I or Ia as photoinitiator and a colorant, the ethylenically unsaturated component is a polyester acrylate binder of higher molecular weight and reactive diluents. Polyester acrylates in the context of the invention are binder systems composed from monomers and oligomers based on acrylated or methacrylated polyesters. Such polyesters are condensation products of polyesterols (polyhydroxyfunctional polyesters) and acrylic or methacrylic acid and anhydrides thereof. Raw material for polyesterols are polyols and polycarboxylic acids or anhydrides.

Examples for polyols are ethylene glycol, 1,2-propane diol, diethylene glycol, 1,4-butane diol, 1,6-hexane diol, neopentyl glycol, trimethyl pentane diol, 1,4-cyclohexane dimethanol, trcyclodecane dimthanol, trimethylolo propane, glycerol, hydroxpivalic acid neopentyl glycol ester, pentaerythritol.

Examples for carboxylic acids, esters and anhydrides are phthalic anhydride, isophthalic acid, therephthalic acid and their lower alkyl ester derivatives, hexahydrophthalic anhydride, tetrahydrophthalic anhydride, hexahydroterephthalic acid, 5-tert-butylisophthalic acid, adipic acid azelaic acid, sebacic acid, decane dicarboxylic acid and their lower alkyl ester derivatives, dimerised fatty acids, trimellitic anhydride, pyromellitic anhydride.

Polyesteracrylates can be further modified for example chlorinated or fluorinated, amine modified or can be functionalized for further crosslinking or reaction with acid or hydroxyl groups. The polyester acrylate in context with the current invention is a binder material with a medium molecular weight ranging from 500 to 6000, preferred are products with a molecular weight from 500 to 3000, more specific from 700 to 2000. The functionality of the polyester acrylate ranges from 1 to 8, preferred are tetra to hexafunctional types. Further examples of polyesterols and polyester acrylates and their preparation can be found in H. Kittel “Lehrbuch der Lehrbuch der Lacke und Beschichtungen” Vol. 2 (“Bindemittel für lösemittelhaltige und lösemiftelfreie Systeme”) 2^{nd} edition, S. Hirzel Verlag Stuttgart—Leipzig, 1998, ISBN 3-7776-0886-6.

Commercial examples for polyester acrylates are EBECRYL 436, EBECRYL 438, EBECRYL 446, EBECRYL 450, EBECRYL 505, EBECRYL 524, EBECRYL 525, EBECRYL 584, EBECRYL 586, EBECRYL 657, EBECRYL 770, EBECRYL 800, EBECRYL 810, EBECRYL 811, EBECRYL 812, EBECRYL 830, EBECRYL 851, EBECRYL 852, EBECRYL 870, EBECRYL 880, EBECRYL 1657, EBECRYL 2047, EBECRYL 531, Laromer PE 55 F, Laromer PE 56 F, Laromer PE 44 F, Laromer LR 8800, Laromer LR 8981, Photomer 5018, Photomer 5029, Jägalux UV 1100, Jägalux UV 1200, Jägalux UV 1300, Setacure AP 578, Setacure AP 576, Setacure AP 578, Setacure AP 579, Syntaalat UV 190, Synocure AC-1007, Synocure AC-1309, Craynor CN 292. Further examples of commercially available polyester acrylate binders can be found in Karsten “Lackrohstoff-Tabellen” 10. edition, Vincentz Verlag Hannover, 2000, ISBN 3-87870-561-1.

Preferred are EBECRYL 800, EBECRYL 810, EBECRYL 830, EBECRYL 885.

The composition in context with the current invention can further contain acrylic monomers i.e. reactive diluents. Reactive diluents are low molecular weight and low viscosity mono to multifunctional ethylenically unsaturated compounds. Examples for monofuntional reactive diluents are butyl acrylate, ethlhexyl acrylate, octyl acrylate, decyl acrylate, isodecyl acrylate lauryl acrylate, stearyl acrylate, hydroxyethyl acrylate, Hydroxyl propyl acrylate, phenoxyethyl acrylate, nonyl phenol ethoxylate monoacrylate, isobornyl acrylate tetrahydrofurfuryl acrylate and methacrylate, cyclohexyl acrylate, dicyclopentenyl acrylate, dicyclopentenyl oxyethylacrylate, propylene glycol monoacrylate and methacrylate, ethoxylated monoacrylate, monofunctional aliphatic urethane acrylates, styrene, vinyl toluene, vinyl acetate.

Examples for multifunctional reactive diluents are trimethylolpropane triacrylate, trimethyllolethane triacrylate, trimethylolpropane trimeth-acrylate, trimethylolethane trimethacrylate, tetramethylene glycol dimethacrylate, triethylene glycol dimethacrylate, tetraethylene glycol diacrylate, pentaerythritol diacrylate, pentaerythritol triacrylate, pentaerythritol tetraacrylate, dipentaerythritol diacrylate, dipentaerythritol triacrylate, dipentaerythritol tetraacrylate, dipentaerythritol pentaacrylate, dipentaerythritol hexaacrylate, tripentaerythritol octaacrylate, pentaerythritol dimethacrylate, pentaerythritol trimethacrylate, dipentaerythritol dimethacrylate, dipentaerythritol tetramethacrylate, tripentaerythritol octamethacrylate, pentaerythritol diitaconate, dipentaerythritol tris-itaconate, dipentaerythritol pentaitaconate, dipentaerythritol hexaitaconate, ethylene glycol diacrylate, 1,3-butanediol diacrylate, 1,3-butanediol dimethacrylate, 1,4-butanediol diitaconate, sorbitol triacrylate, sorbitol tetraacrylate, pentaerythritol-modified triacrylate, sorbitol tetra methacrylate, sorbitol pentaacrylate, sorbitol hexaacrylate, oligoester acrylates and methacrylates, glycerol diacrylate and triacrylate, 1,4-cyclohexane diacrylate, bisacrylates and bismethacrylates of polyethylene glycol with a molecular weight of from 200 to 1500, or mixtures thereof;

Further examples of reactive diluents or monomers can be found in Allen, Johnson, Oldring, Salim “Chemistry & Technology of UV & EB Formulation for Coatings, Inks and Paints”, Vol. 2 (“Prepolymers and Reactive Diluents for UV and EB Curable Formulations”), SITA Technology London, 1991, ISBN 0-947798-10-2.

Preferred are di- or trifunctional aliphatic acrylic esters, more specific hexandiol diacrylate (HDDA) and trimetholpropane triacrylate (TMPTA).

In addition to the photoinitiator the photopolymerizable mixtures may include various additives (e). Examples of these are thermal inhibitors, which are intended to prevent premature polymerization, examples being hydroquinone, hydroquinine derivatives, p-methoxyphenol, β-naphthol or sterically hindered phenols, such as 2,6-di-tert-butyl-p-cresol. In order to increase the stability on storage in the dark it is possible, for example, to use copper compounds, such as copper naphthenate, stearate or octoate, phosphorus compounds, for example triphenylphosphine, tributylphosphine, triethyl phosphite, triphenyl phosphite or tribenzyl phosphite, quaternary ammonium compounds, for example tetramethylammonium chloride or trimethylbenzylammonium chloride, or hydroxylamine derivatives, for example N-diethylhydroxylamine. To exclude atmospheric oxygen during the polymerization it is possible to add paraffin or similar wax-like substances which, being of inadequate solubility in the polymer, migrate to the surface in the beginning of polymerization and form a transparent surface layer which prevents the ingress of air. It is also possible to apply an oxygen-impermeable layer. Light stabilizers which can be added in a small quantity are UV absorbers, for example those of the hydroxyphenylbenzotriazole, hydroxyphenyl-benzophenone, oxalamide or hydroxyphenyl-s-triazine type. These compounds can be used individually or in mixtures, with or without sterically hindered amines (HALS).

Examples of such UV absorbers and light stabilizers are

In addition to those additives it is also possible for the composition to comprise further additives, especially light stabilisers. The nature and amount of such additional additives is governed by the intended use of the coating in question and will be familiar to the person skilled in the art.

As light stabilisers it is possible to add UV absorbers, e.g. those of the hydroxyphenylbenzotriazole, hydroxyphenylbenzophenone, oxalic acid amide or hydroxyphenyl-s-triazine type. Such compounds can be used singly or in the form of mixtures, with or without the use of sterically hindered amines (HALS).

Examples of such UV absorbers and light stabilisers are disclosed in WO 04/074328, page 12, line 9 to page 14, line 23, said disclosure hereby is incorporated by reference.

Further additives known in the art may be added, as for example antistatics, flow improvers and adhesion promoters.

To accelerate the photopolymerization it is possible to add amines, for example triethanolamine, N-methyldiethanolamine, p-dimethylaminobenzoate or Michler's ketone. The action of the amines can be intensified by the addition of aromatic ketones of the benzophenone type. Examples of amines which can be used as oxygen scavengers are substituted N,N-dialkylanilines, as are described in EP 339841. Other accelerators, coinitiators and autoxidizers are thiols, thioethers, disulfides, phosphonium salts, phosphine oxides or phosphines, as described, for example, in EP 438123, in GB 2180358 and in JP Kokai Hei 6-68309.

It is further possible to add chain transfer agents which are customary in the art to the compositions according to the invention. Examples are mercaptanes, amines and benzothiazol.

Photopolymerization can also be accelerated by adding further photosensitizers or coinitiators (d). These are, in particular, aromatic carbonyl compounds, for example benzophenone, thioxanthone, anthraquinone and 3-acylcoumarin derivatives, and also 3-(aroylmethylene)thiazolines, camphor quinone, but also eosine, rhodamine and erythrosine dyes, as well as all compounds which can be used as coinitiators as described above.

Further specific examples of such photosensitizers or coinitiators (d) are

### 1. Thioxanthones

Thioxanthone, 2-isopropylthioxanthone, 2-chlorothioxanthone, 1-chloro-4-propoxythioxanthone, 2-dodecylthioxanthone, 2,4-diethylthioxanthone, 2,4-dimethylthioxanthone, 1-methoxycarbonylthioxanthone, 2-ethoxycarbonylthioxanthone, 3-(2-methoxyethoxycarbonyl)-thioxanthone, 4-butoxycarbonylthioxanthone, 3-butoxycarbonyl-7-methylthioxanthone, 1-cyano-3-chlorothioxanthone, 1-ethoxycarbonyl-3-chlorothioxanthone, 1-ethoxycarbonyl-3-ethoxythioxanthone, 1-ethoxycarbonyl-3-aminothioxanthone, 1-ethoxycarbonyl-3-phenylsulfurylthioxanthone, 3,4-di-[2-(2-methoxyethoxy)ethoxycarbonyl]-thioxanthone, 1,3-dimethyl-2-hydroxy-9H-thioxanthen-9-one 2-ethylhexylether, 1-ethoxycarbonyl-3-(1-methyl-1-morpholinoethyl)-thioxanthone, 2-methyl-4-dimethoxymethyl-thioxanthone, 2-methyl-6-(1,1-dimethoxybenzyl)-thioxanthone, 2-morpholinomethylthioxanthone, 2-methyl-6-morpholinomethylthioxanthone, N-allylthioxanthone-3,4-dicarboximide, N-octylthioxanthone-3,4-dicarboximide, N-(1,1,3,3-tetramethylbutyl)-thioxanthone-3,4-dicarboximide, 1-phenoxythioxanthone, 6-ethoxycarbonyl-2-methoxythioxanthone, 6-ethoxycarbonyl-2-methylthioxanthone, thioxanthone-2-carboxylic acid polyethyleneglycol ester, 2-hydroxy-3-(3,4-dimethyl-9-oxo-9H-thioxanthon-2-yloxy)-N,N,N-trimethyl-1-propanaminium chloride;

### 2. Benzophenones

benzophenone, 4-phenyl benzophenone, 4-methoxy benzophenone, 4,4′-dimethoxy benzophenone, 4,4′-dimethyl benzophenone, 4,4′-dichlorobenzophenone 4,4′-bis(dimethylamino)-benzophenone, 4,4′-bis(diethylamino)benzophenone, 4,4′-bis(methylethylamino)benzophenone, 4,4′-bis(p-isopropylphenoxy)benzophenone, 4-methyl benzophenone, 2,4,6-trimethylbenzophenone, 3-methyl-4′-phenyl-benzophenone, 2,4,6-trimethyl-4′-phenyl-benzophenone, 4-(4-rhethylthiophenyl)-benzophenone, 3,3′-dimethyl-4-methoxy benzophenone, methyl-2-benzoylbenzoate, 4-(2-hydroxyethylthioybenzophenone, 4-(4-tolylthio)benzophenone, 1-[4-(4-benzoyl-phenylsulfanyl)-phenyl]-2-methyl-2-(toluene-4-sulfonyl)-propan-1-one, 4-benzoyl-N,N,N-trimethylbenzenemethanaminium chloride, 2-hydroxy-3-(4-benzoylphenoxy)-N,N,N-trimethyl-1-propanaminium chloride monohydrate, 4-(13-acryloyl-1,4,7,10,13-pentaoxamidecyl)-benzophenone, 4-benzoyl-N,N-dimethyl-N-[2-(1-oxo-2-propenyl)oxy]ethyl-benzenemethanaminium chloride;

### 3. Coumarins

Coumarin 1, Coumarin 2, Coumarin 6, Coumarin 7, Coumarin 30, Coumarin 102, Coumarin 106, Coumarin 138, Coumarin 152, Coumarin 153, Coumarin 307, Coumarin 314, Coumarin 314T, Coumarin 334, Coumarin 337, Coumarin 500, 3-benzoyl coumarin, 3-benzoyl-7-methoxycoumarin, 3-benzoyl-5,7-dimethoxycoumarin, 3-benzoyl-5,7-dipropoxycoumarin, 3-benzoyl-6,8-dichlorocoumarin, 3-benzoyl-6-chloro-coumarin, 3,3′-carbonyl-bis[5,7-di(propoxy)coumarin], 3,3′-carbonyl-bis(7-methoxycoumarin), 3,3′-carbonyl-bis(7-diethylamino-coumarin), 3-isobutyroylcoumarin, 3-benzoyl-5,7-dimethoxy-coumarin, 3-benzoyl-5,7-diethoxy-coumarin, 3-benzoyl-5,7-dibutoxycoumarin, 3-benzoyl-5,7-di(methoxyethoxy)-coumarin, 3-benzoyl-5,7-di(allyloxy)coumarin, 3-benzoyl-7-dimethylaminocoumarin, 3-benzoyl-7-diethylaminocoumarin, 3-isobutyroyl-7-dimethylaminocoumarin, 5,7-dimethoxy-3-(1-naphthoyl)-coumarin, 5,7-diethoxy-3-(1-naphthoyl)-coumarin, 3-benzoylbenzo[f]coumarin, 7-diethylamino-3-thienoylcoumarin, 3-(4-cyanobenzoyl-5,7-dimethoxycoumarin, 3-(4-cyanobenzoyl)-5,7-dipropoxycoumarin, 7-dimethylamino-3-phenylcoumarin, 7-diethylamino-3-phenylcoumarin, the coumarin derivatives disclosed in JP 09-179299-A and JP 09-325209-A, for example 7-[{4-chloro-6-(diethylaminoYS-triazine-2-yl}amino]-3-phenylcoumarin;
4. 3-(arovlmethylene)thiazolines
3-methyl-2-benzoylmethylene-β-naphthothiazoline, 3-methyl-2-benzoylmethylene-benzothiazoline, 3-ethyl-2-propionylmethylene-β-naphthothiazoline;

### 5. Rhodanines

4-dimethylaminobenzalrhodanine, 4-diethylaminobenzalrhodanine, 3-ethyl-5-(3-octyl-2-benzothiazolinylideneyrhodanine, the rhodanine derivatives, formulae [1], [2], [7], disclosed in JP 08-305019A;

### 6. Other Compounds

acetophenone, 3-methoxyacetophenone, 4-phenylacetophenone, benzil, 4,4′-bis(dimethylamino)benzil, 2-acetylnaphthalene, 2-naphthaldehyde, dansyl acid derivatives, 9,10-anthraquinone, anthracene, pyrene, aminopyrene, perylene, phenanthrene, 9-fluorenone, dibenzosuberone, curcumin, xanthone, thiomichler's ketone, α-(4-dimethylaminobenzylidene)ketones, e.g. 2,5-bis(4-diethylaminobenzylidene)cyclopentanone, 2-(4-dimethylaminobenzylideneyindan-1-one, 3-(4-dimethylamino-phenyl)-1-indan-5-yl-propenone, 3-phenylthiophthalimide, N-methyl-3,5-di(ethylthioyphthalimide, N-methyl-3,5-di(ethylthio)-phthalimide, phenothiazine, methylphenothiazine, amines, e.g. N-phenylglycine, ethyl 4-dimethylaminobenzoate, butoxyethyl 4-dimethylaminobenzoate, 4-dimethylaminoacetophenone, triethanolamine, methyldiethanolamine, dimethylaminoethanol, 2-(dimethylamino)ethyl benzoate, poly(propylenegylcol)-4-(dimethylamino)benzoate.

The curing process can be assisted by adding a component which under thermal conditions forms free radicals, for example an azo compound such as 2,2′-azobis(4-methoxy-2,4-dimethylvaleronitrile), a triazene, diazo sulfide, pentazadiene or a peroxy compound, for instance a hydroperoxide or peroxycarbonate, for example t-butyl hydroperoxide, as described for example in EP 245639.

Further customary additives (e), depending on the intended use, are optical brighteners, fillers, wetting agents or levelling assistants.

In order to cure thick and pigmented coatings it is appropriate to add glass microspheres or pulverized glass fibres, as described for example in U.S. Pat. No. 5,013,768.

The choice of additive is made depending on the field of application and on properties required for this field. The additives described above are customary in the art and accordingly are added in amounts which are usual in the respective application.

The invention also provides compositions comprising as component (a) at least one ethylenically unsaturated photopolymerizable compound which is emulsified, dispersed or dissolved in water. Many variants of such radiation-curable aqueous prepolymer dispersions are commercially available.

A prepolymer dispersion is understood as being a dispersion of water and at least one prepolymer dispersed therein. The amount of radiation curable prepolymer or prepolymer mixture, dispersed in the water for example ranges from 20 to 95% by weight, in particular from 30 to 70% by weight. In these compositions the sum of the percentages given for water and prepolymer is in each case 100, with auxiliaries and additives (e.g. emulsifiers) being added in varying quantities depending on the intended use.

The radiation-curable aqueous prepolymer dispersions are known polymeric systems, comprising mono- or polyfunctional ethylenically unsaturated prepolymers, that have an average molecular weight Mₙ (in g/mol) of at least 400, in particular from 500 to 100′000. Prepolymers with higher molecular weights, however, may also be considered depending on the intended application. Use is made, for example, of polyesters having an acid number of not more than 10, of polyethers containing polymerizable C—C double bonds, of hydroxyl-containing reaction products of a polyepoxide, containing at least two epoxide groups per molecule, with at least one α,β-ethylenically unsaturated carboxylic acid, of polyurethane (meth)acrylates and of acrylic copolymers which contain α,β-ethylenically unsaturated acrylic radicals, as are described in EP 12339. Mixtures of these prepolymers can likewise be used. Also suitable are the polymerizable prepolymers described in EP 33896, which are thioether adducts of polymerizable prepolymers having an average molecular weight Mₙ (in g/mol) of at least 600, additionally comprising polymerizable C—C double bonds. Other suitable aqueous dispersions, based on specific alkyl (meth)acrylate polymers, are described in EP 41125.

Further additives (e) which may be included in these radiation-curable aqueous prepolymer dispersions are dispersion auxiliaries, emulsifiers, antioxidants, light stabilizers, fillers, for example talc, gypsum, silicic acid, rutile, carbon black, zinc oxide, iron oxides, reaction accelerators, levelling agents, lubricants, wetting agents, thickeners, flatting agents, antifoams and other auxiliaries customary in paint technology. Suitable dispersion auxiliaries are water-soluble organic compounds which are of high molecular mass and contain polar groups, examples being polyvinyl alcohols, polyvinylpyrrolidone or cellulose ethers. Emulsifiers which can be used are nonionic emulsifiers and, if desired, ionic emulsifiers as well.

In certain cases it may be of advantage to use mixtures of two or more the photoinitiators of the formula I. It is of course also possible to use mixtures with known photoinitiators of another type (b1) for example mixtures with camphor quinone; benzophenone, benzophenone derivatives, such as 2,4,6-trimethylbenzophenone, 2-methylbenzophenone, 3-methylbenzophenone, 4-methylbenzophenone, 2-methoxycarbonylbenzophenone 4,4′-bis(chloromethyl)benzophenone, 4-chlorobenzophenone, 4-phenylbenzophenone, 3,3′-dimethyl-4-methoxy-benzophenone, [4-(4-methylphenylthio)phenyl]-phenylmethanone, methyl-2-benzoylbenzoate, 3-methyl-4′-phenylbenzophenone, 2,4,6-trimethyl-4′-phenylbenzophenone, 4,4′-bis(dimethylamino)benzophenone, 4,4′-bis(diethylamino)benzophenone; ketal compounds, as for example benzildimethylketal (IRGACURE® 651); acetophenone, acetophenone derivatives, for example α-hydroxycycloalkyl phenyl ketones or 2-hydroxy-2-methyl-1-phenyl-propanone (DAROCUR® 1173), 1-hydroxy-cyclohexyl-phenyl-ketone (IRGACURE ® 184)1-(4-dodecylbenzoyl)-1-hydroxy-1-methyl-ethane, 1-(4-isopropylbenzoyl)-1-hydroxy-1-methyl-ethane, 1-[4-(2-Hydroxyethoxy)-phenyl]-2-hydroxy-2-methyl-1-propan-1-one (IRGACURE ® 2959); 2-Hydroxy-1-{4-[4-(2-hydroxy-2-methyl-propionyl)-benzyl]-phenyl}-2-methyl-propan-1-one (IRGACURE®127); 2-Hydroxy-1-{4-[4-(2-hydroxy-2-methyl-propionyl)-phenoxy]-phenyl}-2-methyl-propan-1-one; dialkoxyacetophenones, α-hydroxy- or α-aminoacetophenones, e.g. (4-methylthiobenzoyl)-1-methyl-1-morpholinoethane (IRGACURE® 907), (4-morpholinobenzoyl)-1-benzyl-1-dimethylaminopropane (IRGACURE® 369), (4-morpholinobenzoyl)-1-(4-methylbenzyl)-1-dimethylaminopropane (IRGACURE® 379), (4-(2-hydroxyethyl)aminobenzoyl)-1-benzyl-1-dimethylaminopropane), (3,4-dimethoxybenzoyl)-1-benzyl-1-dimethylaminopropane; 4-aroyl-1,3-dioxolanes, benzoin alkyl ethers and benzil ketals, e.g. dimethyl benzil ketal, phenylglyoxalic esters and derivatives thereof, e.g. oxophenyl-acetic acid 2-(2-hydroxy-ethoxy)-ethyl ester, dimeric phenylglyoxalic esters, e.g. oxophenyl-acetic acid 1-methyl-2-[2-(2-oxo-2-phenyl-acetoxy)-propoxy]-ethyl ester (IRGACURE® 754); oximeesters, e.g. 1,2-octanedione 1-[4-(phenylthio)phenyl]-2-(O-benzoyloxime) (IRGACURE® OXE01), ethanone 1-[9-ethyl-6-(2-methylbenzoyl)-9H-carbazol-3-yl]-1-(O-acetyloxime) (IRGACURE® OXE02), 9H-thioxanthene-2-carboxaldehyde 9-oxo-2-(O-acetyloxime), peresters, e,g. benzophenone tetracarboxylic peresters as described for example in EP 126541, monoacyl phosphine oxides, e.g. (2,4,6-trimethylbenzoyl)diphenylphosphine oxide (DAROCUR® TPO), ethyl (2,4,6 trimethylbenzoyl phenyl)phosphinic acid ester; bisacylphosphine oxides, e.g. bis(2,6-dimethoxy-benzoyl)-(2,4,4-trimethyl-pentyl)phosphine oxide, bis(2,4,6-trimethylbenzoyl)-phenylphosphine oxide (IRGACURE® 819), bis(2,4,6-trimethylbenzoyl-2,4-dipentoxyphenylphosphine oxide, trisacylphosphine oxides, halomethyltriazines, e.g. 2-[2-(4-methoxy-phenyl)-vinyl]-4,6-bistrichloromethyl-[1,3,5]triazine, 2-(4-methoxy-phenyl)-4,6-bis-trichloromethyl-[1,3,5]triazine, 2-(3,4-dimethoxy-phenyl)-4,6-bis-trichloromethyl-[1,3,5]-triazine, 2-methyl-4,6-bistrichloromethyl-[1,3,5]triazine, hexaarylbisimidazole/coinitiator systems, e.g. ortho-chlorohexaphenyl-bisimidazole combined with 2-mercaptobenzthiazole, ferrocenium compounds, or titanocenes, e.g. bis(cyclopentadienyl)-bis(2,6-difluoro-3-pyrryl-phenyl)titanium (IRGACURE®784). Further, borate compounds can be used as coinitiators.

The photopolymerizable compositions generally comprise 0.05 to 15% by weight, preferably 0.1 to 5% by weight, of the curing agent (b), based on the total composition. The amount refers to the sum of all photoinitiators added, if mixtures of initiators are employed. Accordingly, the amount either refers to the photoinitiator (b) or the photoinitiators (b)+(b1).

The photopolymerizable compositions can be used for various purposes, for example as printing ink, e.g. as screen printing ink, ink for flexoprinting or offsetprinting, sheet-fed printing, as a colored finish, for example for wood or metal, as powder coating, as a coating material, inter alia for paper, wood, metal or plastic, as a daylight-curable coating for the marking of buildings and roadmarking, for photographic reproduction techniques, for holographic recording materials, for image recording techniques or to produce printing plates which can be developed with organic solvents or with aqueous alkalis, for producing masks for screen printing, as adhesives, as pressure-sensitive adhesives, as laminating resins, as photoresits, e.g. etch resists, electroplating resists, or permanent resists, both liquid and dry films, as photostructurable dielectricum and as solder masks for electronic circuits, as resists to manufacture color filters for any type of display applications or to generate structures in the manufacturing process of plasma-display panels and electroluminescence displays, for the production of optical switches, optical lattices (interference lattice), light circuits, for producing three-dimensional articles by mass curing (UV curing in transparent moulds) or by the sterelithography technique, as is described, for example, in U.S. Pat. No. 4,575,330, to produce composite materials (for example styrenic polyesters, which may, if desired, contain glass fibres and/or other fibres and other auxiliaries) and other thick-layered compositions, for coating or sealing electronic components and chips, as gel coats or as coatings for optical fibres.

The compositions according to the invention are further suitable for the production of medical equipment, auxiliaries or implants, e.g. contact lenses.

Further the compositions according to the invention are suitable for the preparation of gels with thermotropic properties, as for example described in DE 19700064 and EP 678534.

The compositions according to the invention can also be used in dry paint film, as for example described in Paint&Coatings Industry, April 1997, 72 or Plastics World, vol. 54, no. 7, p48(5).

The curing agent of formula I or Ia may additionally be employed as initiators for fixing dyes on organic materials.

In coating materials, use is frequently made of mixtures of a prepolymer with polyunsaturated monomers, which may additionally include a monounsaturated monomer as well. It is the prepolymer here which primarily dictates the properties of the coating film, and by varying it the skilled worker is able to influence the properties of the cured film. The polyunsaturated monomer functions as a crosslinking agent which renders the film insoluble. The monounsaturated monomer functions as a reactive diluent, which is used to reduce the viscosity without the need to employ a solvent.

Unsaturated polyester resins are usually used in two-component systems together with a monounsaturated monomer, preferably with styrene. For photoresists, specific one-component systems are often used, for example polymaleimides, polychalcones or polyimides, as described in DE 2308830.

The compositions according to the invention can also be used as radiation-curable powder coatings. The powder coatings can be based on solid resins and monomers containing reactive double bonds, for example maleates, vinyl ethers, acrylates, acrylamides and mixtures thereof. A free-radically UV-curable powder coating can be formulated by mixing unsaturated polyester resins with solid acrylamides (for example methyl methylacrylamidoglycolate) and a novel free-radical photoinitiator, such formulations being as described, for example, in the paper “Radiation Curing of Powder Coating”, Conference Proceedings, Radtech Europe 1993 by M. Wittig and Th. Gohmann. The powder coatings can also contain binders, as are described, for example, in DE 4228514 and in EP 636669. Free-radically UV-curable powder coatings can also be formulated by mixing unsaturated polyester resins with solid acrylates, methacrylates or vinyl ethers and with a novel photoinitiator (or photoinitiator mixture). The powder coatings may also comprise binders as are described, for example, in DE 4228514 and in EP 636669. The procedure normally comprises electrostatic or tribostatic spraying of the powder onto the substrate, for example metal or wood, melting of the powder by heating, and, after a smooth film has formed, radiation-curing of the coating with ultraviolet and/or visible light, using for example medium-pressure mercury lamps, metal halide lamps or xenon lamps. A particular advantage of the radiation-curable powder coatings over their heat-curable counterparts is that the flow time after melting the powder particles can be delayed in order to ensure the formation of a smooth, high-gloss coating. In contrast to heat-curable systems, radiation-curable powder coatings can be formulated to melt at lower temperatures without the unwanted effect of shortening their lifetime. For this reason, they are also suitable as coatings for heat-sensitive substrates, for example wood or plastics.

In addition to the photoinitiator, the powder coating formulations may also include UV absorbers. Appropriate examples are listed above in sections 1.-8.

The novel photocurable compositions are suitable, for example, as coating materials for substrates of all kinds, for example wood, textiles, paper, ceramics, glass, plastics such as polyesters, polyethylene terephthalate, polyolefins or cellulose acetate, especially in the form of films, and also metals such as Al, Cu, Ni, Fe, Zn, Mg or Co and GaAs, Si or SiO₂ to which it is intended to apply a protective layer or, by means of imagewise exposure, to generate an image.

Coating of the substrates can be carried out by applying to the substrate a liquid composition, a solution or a suspension. The choice of solvents and the concentration depend principally on the type of composition and on the coating technique. The solvent should be inert, i.e. it should not undergo a chemical reaction with the components and should be able to be removed again, after coating, in the course of drying. Examples of suitable solvents are ketones, ethers and esters, such as methyl ethyl ketone, isobutyl methyl ketone, cyclopentanone, cyclohexanone, N-methylpyrrolidone, dioxane, tetrahydrofuran, 2-methoxyethanol, 2-ethoxyethanol, 1-methoxy-2-propanol, 1,2-dimethoxyethane, ethyl acetate, n-butyl acetate and ethyl 3-ethoxypropionate.

The solution is applied uniformly to a substrate by means of known coating techniques, for example by spin coating, dip coating, knife coating, curtain coating, brushing, spraying, especially by electrostatic spraying, and reverse-roll coating, and also by means of electrophoretic deposition. It is also possible to apply the photosensitive layer to a temporary, flexible support and then to coat the final substrate, for example a copper-clad circuit board, by transferring the layer via lamination.

The quantity applied (coat thickness) and the nature of the substrate (layer support) are dependent on the desired field of application. The range of coat thicknesses generally comprises values from about 0.1 μm to more than 100 μm, for example 0.1 to 300 μm, e.g. 0.1-200 μm, 0.1-150 μm, 10-300 μm, 10-200 μm, 50-300 μm, 50-200 μm, 100-300 μm, 100-200 μm or 120-300 μm.

Preferred are wet coating thicknesses of more than 100 μm, e.g. 100-300 μm, 100-200 μm or 120-300 μm.

Accordingly to apply and cure high thicknesses of film, the amount of colorant can be increased up to 60%, preferably 10 to 40% to increase hiding power. Thicknesses applied with higher pigment loading range between 0.1 to 200 μm, for example between 0.1 to 150 μm, 10 to 100 μm, 20 to 60 μm.

Photocuring is of great importance for printings, since the drying time of the ink is a critical factor for the production rate of graphic products, and should be in the order of fractions of seconds. UV-curable inks are particularly important for screen printing and offset inks.

The compositions are suitable also for producing printing plates. This application uses, for example, mixtures of soluble linear polyamides or styrene/butadiene and/or styrene/isoprene rubber, polyacrylates or polymethyl methacrylates containing carboxyl groups, polyvinyl alcohols or urethane acrylates with photopolymerizable monomers, for example acrylamides and/or methacrylamides, or acrylates and/or methacrylates, and a photoinitiator. Films and plates of these systems (wet or dry) are exposed over the negative (or positive) of the printed original, and the uncured parts are subsequently washed out using an appropriate solvent or aqueos solutions.

Another field where photocuring is employed is the coating of metals, in the case, for example, of the coating of metal plates and tubes, cans or bottle caps, and the photocuring of polymer coatings, for example of floor or wall coverings based on PVC.

Examples of the photocuring of paper coatings are the colourless varnishing of labels, record sleeves and book covers.

Also of interest is the use of the novel compositions for curing shaped articles made from composite compositions. The composite compound consists of a self-supporting matrix material, for example a glass fibre fabric, or alternatively, for example, plant fibres [cf. K.-P. Mieck, T. Reussmann in Kunststoffe 85 (1995), 366-370], which is impregnated with the photocuring formulation. Shaped parts comprising composite compounds, when produced using the novel compounds, attain a high level of mechanical stability and resistance. The novel compounds can also be employed as photocuring agents in moulding, impregnating and coating compositions as are described, for example, in EP 7086. Examples of such compositions are gel coat resins, which are subject to stringent requirements regarding curing activity and yellowing resistance, and fibre-reinforced mouldings, for example, light diffusing panels which are planar or have lengthwise or crosswise corrugation. Techniques for producing such mouldings, such as hand lay-up, spray lay-up, centrifugal casting or filament winding, are described, for example, by P. H. Selden in “Glasfaserverstärkte Kunststoffe”, page 610, Springer Verlag Berlin-Heidelberg—New York 1967. Examples of articles which can be produced by these techniques are boats, fibre board or chipboard panels with a double-sided coating of glass fibre-reinforced plastic, pipes, containers, etc. Further examples of moulding, impregnating and coating compositions are UP resin gel coats for mouldings containing glass fibres (GRP), such as corrugated sheets and paper laminates. Paper laminates may be based on urea resins or melamine resins. Prior to production of the laminate, the gel coat is produced on a support (for example a film). The novel photocurable compositions can also be used for casting resins or for embedding articles, for example electronic components, etc. Curing usually is carried out using medium-pressure mercury lamps as are conventional in UV curing. However, there is also particular interest in less intense lamps, for example of the type TL 40W/03 or TL40W/05. The intensity of these lamps corresponds approximately to that of sunlight. It is also possible to use direct sunlight for curing. A further advantage is that the composite composition can be removed from the light source in a partly cured, plastic state and can be shaped, with full curing taking place subsequently.

The photosensitivity of the novel compositions can extend in general from about 150 nm to 600 nm, for example 190-600 nm, (UV-vis region). Suitable radiation is present, for example, in sunlight or light from artificial light sources. Consequently, a large number of very different types of light sources are employed. Both point sources and arrays (“lamp carpets”) are suitable. Examples are carbon arc lamps, xenon arc lamps, low-, medium-, high- and super high-pressure mercury lamps, possibly with metal halide dopes (metal-halogen lamps), microwave-stimulated metal vapour lamps, excimer lamps, superactinic fluorescent tubes, fluorescent lamps, argon incandescent lamps, electronic flashlights, photographic flood lamps, electron beams and X-rays. The distance between the lamp and the substrate to be exposed in accordance with the invention may vary depending on the intended application and the type and output of lamp, and may be, for example, from 2 cm to 150 cm. Laser light sources, for example excimer lasers, such as F₂ excimer lasers at 157 nm exposure, KrF excimer lasers for exposure at 248 nm and ArF excimer lasers for exposure at 193 nm are also suitable. Lasers in the visible region can also be employed. Alternatively, the actinic radiation is provided by light emitting diodes (LED), e.g. UV light emitting diodes (UV-LED). Said LEDs allow instant on and off switching of the radiation source. Further, UV-LEDs generally have a narrow wavelength distribution and offer the possibility to customize the peak wavelength and also provide an efficient conversion of electric energy to UV radiation.

The invention therefore also provides a process for the curing colored compositions, comprising applying a colored composition as defined above on at least one surface of a substrate and irradiating the composition with light in the range from 200 to 600 nm; as well as a process, wherein the composition, is applied to the substrate in a thickness of 0.1 to 300 μm; and subsequently is irradiated with light in the wavelength range from 200 to 600 nm.

The invention additionally pertains the use of compounds of a photoinitiator compound of the phenylglyoxylate type as curing agent for the curing of colored UV-curable compositions comprising a uv- and short-vis-absorbing colorant by irradiation with light in the wavelength range from 200 to 600 nm.

Short-wavelength V is is considered to be radiation in the area of 380-500 nm

The invention further provides a coated substrate which is coated on at least one surface with a composition as described above.

Another subject of the invention is the use of a composition as described above, for the preparation of pigmented surface coatings, printing inks, screen printing inks, offset printing inks, flexographic printing inks, powder coatings, printing plates, adhesives, composite materials, gel coats, glass-fibre cable coatings, screen printing stencils, resist materials, colour filters, of three-dimensional objects by means of stereolithography, of photographic reproductions or image recording material as well as a process for the preparation of pigmented surface coatings, printing inks, screen printing inks, offset printing inks, flexographic printing inks, powder coatings, printing plates, adhesives, composite materials, gel coats, glass-fibre cable coatings, screen printing stencils, resist materials, colour filters, of three-dimensional objects by means of stereolithography, of photographic reproductions or image recording material.

Further subjects of the invention are a process for the photopolymerization of compounds containing ethylenically unsaturated double bonds, which comprises irradiating a composition as described above, comprising a compound of the formula (Ib), (1), (2), (3) or (4) as photoinitiator with electromagnetic radiation in the range from 150 to 600 nm, or with electron beam or with X-rays; as well as the use of a compound of the formula (Ib) as defined above or a compound (1), (2), (3) or (4) as defined in claim 15 as a photoinitiator; the use of a composition as described above, comprising a compound of the formula (Ib), (1), (2), (3) or (4) as photoinitiator for producing pigmented and nonpigmented paints and varnishes, powder coatings, printing inks, printing plates, adhesives, pressure sensitive adhesives, dental compositions, gel coats, photoresists, electroplating resists, etch resists, both liquid and dry films, solder resists, resists to manufacture color filters, resists to generate structures in the manufacturing processes of plasma-display panels, electroluminescence displays and LCD, spacers for LCD, for holographic data storage (HDS), as composition for encapsulating electrical and electronic components, for producing magnetic recording materials, micromechanical parts, waveguides, optical switches, plating masks, etch masks, colour proofing systems, glass fibre cable coatings, screen printing stencils, for producing three-dimensional objects by means of stereolithography, as image recording material, for holographic recordings, microelectronic circuits, decolorizing materials, decolorizing materials for image recording materials, for image recording materials using microcapsules, as a photoresist material for a UV and visible laser direct imaging system, as a photoresist material used for forming dielectric layers in a sequential build-up layer of a printed circuit board.

Another aspect of the invention is a process for producing pigmented and nonpigmented paints and varnishes, powder coatings, printing inks, printing plates, adhesives, pressure sensitive adhesives, dental compositions, gel coats, photoresists for electronics, electroplating resists, etch resists, both liquid and dry films, solder resists, resists to manufacture color filters for a variety of display applications, resists to generate structures in the manufacturing processes of plasma-display panels, electroluminescence displays and LCD, spacers for LCD, for holographic data storage (HDS), as composition for encapsulating electrical and electronic components, for producing magnetic recording materials, micromechanical parts, waveguides, optical switches, plating masks, etch masks, colour proofing systems, glass fibre cable coatings, screen printing stencils, for producing three-dimensional objects by means of stereolithography, as image recording material, for holographic recordings, microelectronic circuits, decolorizing materials, decolorizing materials for image recording materials, for image recording materials using microcapsules, as a photoresist material for a UV and visible laser direct imaging system, as a photoresist material used for forming dielectric layers in a sequential build-up layer of a printed circuit board: as well as a coated substrate which is coated on at least one surface with a composition comprising a photoinitiator of the formula (Ib), (1), (2), (3) or (4) as described above.

The compositions according to the present invention are in particular suitable in coatings applications, especially for colored coatings with higher coating thicknesses or increased colorant content.

The examples which follow illustrate the invention in more detail, without restriciting the scope said examples only. Parts and percentages are, as in the remainder of the description and in the claims, by weight, unless stated otherwise. Where alkyl radicals having more than three carbon atoms are referred to in the examples without any mention of specific isomers, the n-isomers are meant in each case.

### PREPARATION EXAMPLES

### Example 1

### Preparation of

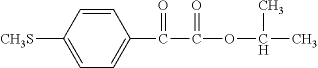

3 g (0.014 mol) of

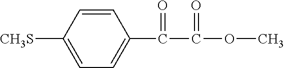

are dissolved in 40 g (0.67 mol) isopropanol. The reaction mixture is heated up to a temperature of 75° C. At a temperature of 40° C. four drops of dibutyl-tin-dilaureate as transesterification catalyst are added. The reaction mixture is kept over night at a temperature of 75° C. until all starting material is consumed. The reaction mixture was cooled down to room temperature, diluted with methylene chloride and twice extracted with water. The organic phase was dried over MgSO₄ and the solvent is evaporated. The yellow oil residue is purified by column chromatography with heptane/toluene (1:1) as eluant mixture. 2.4 g of the title product are obtained in form of yellow oil (yield: 72%). (The starting material compound

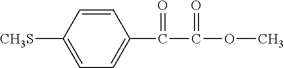

is synthesized as described in WO 9833761A1).

### Examples 2-6

The compounds of the examples 2-6 are prepared according to the method as described in example 1, using the corresponding appropriate alcohols for the transesterification reaction. The compounds and their physical data are collected in table 1.

TABLE 1

| | | UV | | yield |
|---|---|---|---|---|
| | | [(λmax (nm), ε-value | | [% of |
| Ex. | compound | (l/mol · cm)] in CH₃CN | mp (° C.) | th.] |
| 1 | 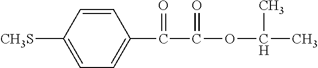 | 22.100 at 326 nm | oil (yellow) | 72 |
| 2 | 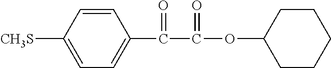 | 22.500 at 326 nm | oil (yellow) | 81 |
| 3 | 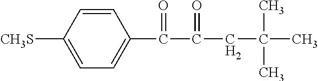 | 22.600 at 326 nm | oil (yellow) | 70 |
| 4 | 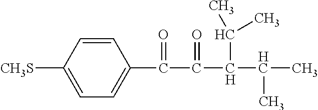 | 22.550 at 325 nm | oil (yellow) | 24 |
| 5 | 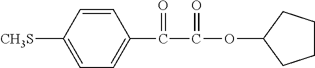 | 22.000 at 325 nm | oil (yellow) | 44 |
| 6 | 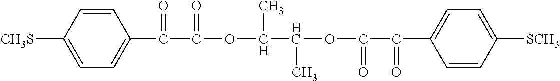 | 40.600 at 327 nm | 112-113 | 28 |

### Example 7

### Preparation of

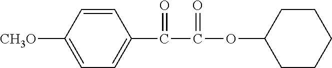

2 g (0.01 mol) of

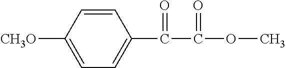

are dissolved in 15 g (0.15 mol) cyclohexanol and 10 mg of dibutyl-tin-dilaureate as transesterification catalyst. The reaction mixture is heated up to a temperature of 120° C. The reaction mixture is kept over night at a temperature of 120° C. until all starting material is consumed. The reaction mixture is cooled down to room temperature, diluted with methylene chloride and twice extracted with water. The organic phase is dried over MgSO₄ and the solvent is evaporated. The yellow oil residue is purified by column chromatography with heptane/toluene (1:1) as eluant mixture. 1.68 g of the title product are obtained in form of yellow oil (yield: 62%).
(The starting material compound

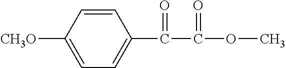

is synthesized as described in WO 9833761A1).

### Examples 8-9

The compounds of the examples 8 and 9 are prepared according to the method as described in example 7, using the corresponding appropriate alcohols for the transesterification reaction. The compounds and their physical data are collected in table 2.

TABLE 2

| | | UV | | yield |
|---|---|---|---|---|
| | | [(λmax (nm), ε-value | | [% of |
| Ex. | compound | (l/mol · cm)] in CH₃CN | mp (° C.) | th.] |
| 7 | 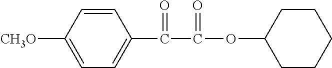 | 18.850 at 292 nm | oil (yellow) | 62 |
| 8 | 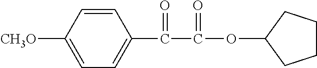 | 24.600 at 288 nm | oil (yellow) | 58 |
| 9 | 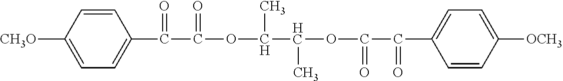 | 33.500 at 294 nm | 75-80 | 18 |

### APPLICATION EXAMPLES

In the application examples, the following photoinitiators are employed:

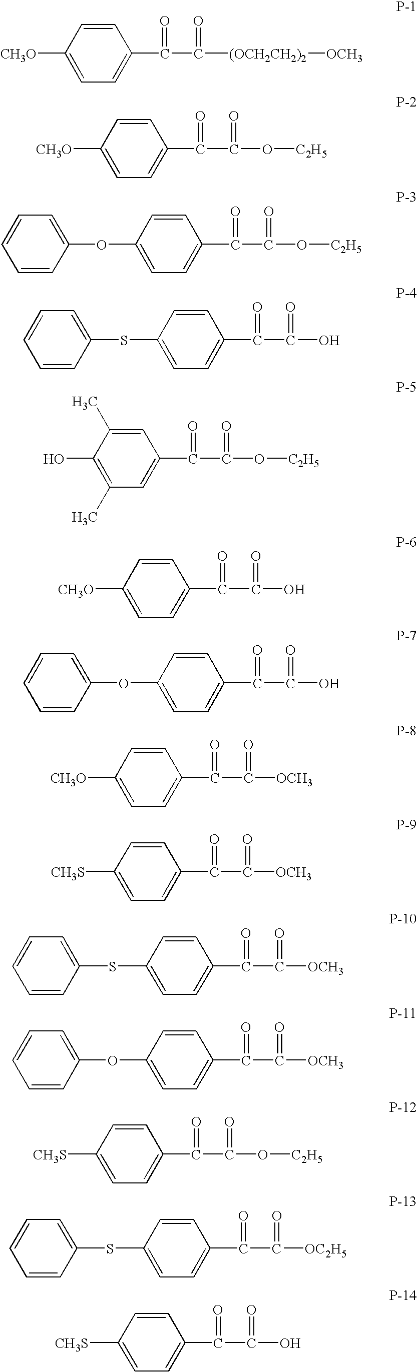

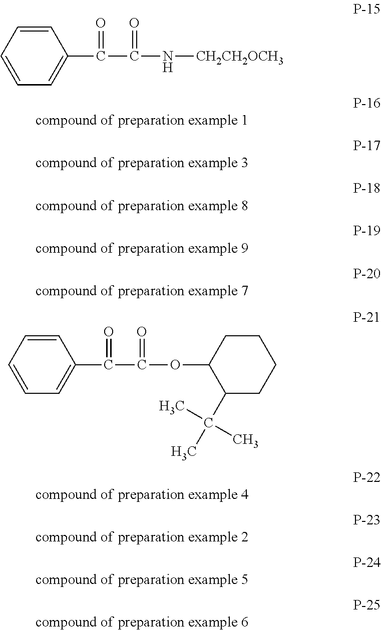

### Application Example 1

### Curing of a Yellow Pigmented Composition

### Formulation:

| | |
|---|---|
| 54.60 parts | of a hexafunctional polyesteracrylate (EBECRYL ® 830, |
| | provided by Cytec Surface Specialties) |
| 13.65 parts | of hexanediol diacrylate (reactive diluent, provided by |
| | Cytec Surface Specialties) |
| 13.65 parts | of trimethylolpropane triacrylate (reactive diluent, provided |
| | by Cytec Surface Specialties) |
| 9.10 parts | of an organic yellow pigment (P.Y. 128) |
| | (CROMOPHTAL ® Yellow 8GN, provided by |
| | Ciba Specialty Chemicals) |
| 9.01 parts | of a polymeric dispersant (EFKA ® 4050, provided by |
| | Ciba Specialty Chemicals) |

Pigment, reactive diluents and dispersant are premixed using a tooth wheel until the pigment is wetted. EBECRYL® 830 is added and premixing is continued for 15 min. 200 parts of glass beads (2 mm) are added and the formulation is dispersed for 60 min using a pearl mill, followed by separation of the formulation and the glass beads.

The photoinitiators to be tested are used in an equimolar amount of radicals formed. The photoinitators are preweighed and 10 g of the formulation are added and stirred in a closed container at 60° C. for 30 min using a magnet stirrer. The tested photoinitiators are listed in table 1. The samples are applied on white pre-coated aluminum coil coat panels using slit coaters with increasing slit sizes (30 to 130 μm in 10 μm steps, then 150 and 200 μm slits).

The slit size is considered the applied wet film thickness (WFT). After application, the samples are cured on Fusion UV-curing equipment using 2 Hg Bulbs at 100% (120 W/cm), aluminum reflectors and a line speed of 2.5 m/min. After curing, the panels are bent in panel middie crossways to cause the coating to crack and delaminate from the substrate. The reverse side of the coating is evaluated by touch for through-curing, dry to touch means good through-curing, tacky or liquid reverse side means poor through-curing. Determined is the maximum curable wet film thickness (WFTₘₐₓ) as measure for through curing properties of the photoinitiators. The maximum applicable slit size is the maximum curable wet film thickness (WFTₘₐₓ). A deviation of +/−10 μm WFTₘₐₓ is considered.

The tested compounds as well as the results are collected in table 3.

TABLE 3

| | Weight | | | Weight | |
|---|---|---|---|---|---|
| Photo- | [g/100 g | WFTₘₐₓ | Photo- | [g/100 g | WFTₘₐₓ |
| initiator | formulation] | [μm] | initiator | formulation] | [μm] |
| P-1 | 4.23 | 150 | P-2 | 3.12 | 130 |
| P-3 | 4.05 | 150 | P-4 | 3.87 | 130 |
| P-5 | 3.33 | 130 | P-6 | 2.70 | 130 |
| P-7 | 3.63 | 130 | P-8 | 2.91 | 120 |
| P-9 | 3.15 | 150 | P-10 | 4.08 | 150 |
| P-11 | 3.84 | 100 | P-12 | 3.36 | 150 |
| P-13 | 4.30 | 150 | P-14 | 2.94 | 120 |
| P-15 | 3.10 | 150 | P-16 | 3.60 | 150 |
| P-17 | 4.00 | 110 | P-18 | 3.72 | 120 |
| P-19 | 3.11 | 110 | P-20 | 3.93 | 120 |
| P-21 | 4.33 | 120 | P-22 | 4.42 | 150 |
| P-24 | 3.97 | 100 | | | |

### Application Example 2

### Curing of Full Shades and White Reductions

### 2.1 Formulations

### Basic Formulation Components are:

EBECRYL® 830, a hexafunctional polyester acrylate, provided by Cytec Surface Specialties; hexandiol diacrylate (HDDA), a recafive diluent, provided by Cytec Surface Specialties; trimethylolpropane triacrylate (TMPTA), a recative diluent, provided by Cytec Surface Specialties; EFKA 4050, a polymeric dispersant, provided by Ciba Speciatly Chemicals; Disperbyk 110, a dispersing agent, provided by BYK-Chemie.

### List of Pigments:

CROMOPHTAL® Yellow 8GN (P.Y. 128); IRGAZIN® Yellow 2093 (P.Y. 184); IRGAZIN® DPP Orange RA (P.O. 73); IRGAZIN® Red 2030; IRGALITE® Red 3RS (P.R. 112); IRGAZIN® Green 2180 (P.G. 007), all provided by Ciba Speciatly Chemicals; Special Black 4, provided by Degussa; Kronos 2310, provided by Kronos.

Formulations for Full Shades (FS):

| FS-1 | “CROMOPHTAL ® Yellow 8 GN”: | |
|---|---|---|
| | the formulation corresponds to the | |
| | one as described in example 1 | |
| FS-2 | “IRGAZIN ® Yellow 2093”: | |
| | 10.00 parts | IRGAZIN ® Yellow 2093 |
| | 15.00 parts | HDDA |
| | 14.30 parts | TMPTA |
| | 0.70 parts | EFKA ® 4050 |
| | 60.00 parts | EBECRYL ® 830 |
| FS-3 | “IRGAZIN ® DPP Orange RA”: | |
| | 9.77 parts | IRGAZIN ® DPP Orange RA |
| | 14.66 parts | HDDA |
| | 14.66 parts | TMPTA |
| | 2.28 parts | EFKA ® 4050 |
| | 58.63 parts | EBECRYL ® 830 |
| FS-4 | “IRGAZIN ® red 2030”: | |
| | 9.72 parts | IRGAZIN ® red 2030 |
| | 14.58 parts | HDDA |
| | 14.58 parts | TMPTA |
| | 2.81 parts | EFKA ® 4050 |
| | 58.31 parts | EBECRYL ® 830 |
| FS-5 | “IRGALITE ® Red 3RS”: | |
| | 10.00 parts | IRGALITE ® Red 3RS |
| | 14.50 parts | HDDA |
| | 14.00 parts | TMPTA |
| | 4.50 parts | EFKA ® 4050 |
| | 57.00 parts | EBECRYL ® 830 |
| FS-6 | “IRGAZIN ® Green 2180” (Paste) | |
| | 10.00 parts | IRGAZIN ® Green 2180 |
| | 15.00 parts | HDDA |
| | 14.50 parts | TMPTA |
| | 2.50 parts | EFKA ® 4050 |
| | 58.00 parts | EBECRYL ® 830 |
| FS-7 | “Special Black 4”: | |
| | 3.00 parts | Special Black 4 |
| | 17.00 parts | HDDA |
| | 16.00 parts | TMPTA |
| | 2.00 parts | EFKA ® 4050 |
| | 62.00 parts | EBECRYL ® 830 |
| FS-8 | “Kronos 2310”: | |
| | 30.00 parts | IRGALITE ® Red 3RS |
| | 9.50 parts | HDDA |
| | 12.00 parts | TMPTA |
| | 1.50 parts | DISPERBYK ® 110 |
| | 47.00 parts | EBECRYL ® 830 |

The pigment, HDDA, TMPTA, dispersant and half the amount of EBECRYL® 830 are cornbined with 230 g glass beads (Ø 2 mm) and dispersed on a LAU Disperser for 60 min, except for FS-6, IRGAZIN® Green 2180, which is dispersed for 120 min. The remaining amount of EBECRYL® 830 is added to the formulation and stirred in using a dissolver unit at low speed before separating glass beads and formulation.

IRGAZIN® Green 2180 (Paste) is combined with Kronos 2310 (FS) in a pigment ratio of 50:50 and stirred with a magnet stirring device until well mixed. The paint is now called FS 6 IRGAZIN® Green 2180 (50:50).

### Formulations for White Reductions (WR):

The WR formulations are prepared by stirring the white formulation Kronos 2310 (FS-8) with the corresponding color pigmented full shade. White reductions with IRGAZIN® Yellow 2093, IRGALITE® Red 3RS, IRGAZIN® Green 2180 are prepared to match ⅓ international standard depth (ISD). The white reductions are prepared according to the following pigment ratios:

| | | | Pigment |
|---|---|---|---|
| | | Pigment | ratio |
| White | | ratio colored | Kronos |
| Reduction | Colored pigment | pigment | 2310 |
| WR-1 | CROMOPHTAL ® Yellow 8 GN | 10.00 | 90.00 |
| WR-2 | IRGAZIN ® Yellow 2093, | 1.00 | 1.00 |
| | ⅓ ISD | | |
| WR-3 | IRGAZIN ® DPP Orange RA | 10.00 | 90.00 |
| WR-4 | IRGAZIN ® Red 2030 | 10.00 | 90.00 |
| WR-5 | IRGALITE ® Red 3RS, ⅓ ISD | 1.00 | 4.50 |
| WR-6 | IRGAZIN ® Green 2180, | 1.00 | 1.30 |
| | ⅓ ISD | | |
| WR-7 | Special Black 4 | 6.00 | 94.00 |

### Paint Samples:

| Full Shade formulations: | | | White reduced formulations: | | |
|---|---|---|---|---|---|
| | Photo- | Photo- | | Photo- | Photo- |
| | inititator | inititator | | inititator | inititator |
| | P-9 [% by | P-10 [% by | | P-9 [% by | P-10 [% by |
| Paint | weight] | weight] | Paint | weight] | weight] |
| FS-1 | 3.15 | 4.08 | WR-1 | 3.15 | 4.08 |
| FS-2 | 3.00 | — | WR-2 | 3.00 | — |
| FS-3 | — | 4.08 | WR-3 | — | 4.08 |
| FS-4 | — | 4.08 | WR-4 | — | 4.08 |
| FS-5 | 3.00 | — | WR-5 | 3.00 | — |
| FS-6 | 3.00 | — | WR-6 | 3.00 | — |
| FS-7 | 3.00 | — | WR-7 | 3.00 | — |

10 g paint samples are prepared by pre-weighing the photoinitiator then adding the formulation. The samples are then stirred with a magnet stirring device at 60° C. for 30 min.

### 2.2 Experimental and Results

Application of the paint, curing and determination of the maximum curable wet film thickness is performed as described in example 1. The results are collected in table 4.

TABLE 4

| | WFTₘₐₓ | WFTₘₐₓ |
|---|---|---|
| | [μm] | [μm] |
| | with | with |
| Paint | P-9 | P-7 |
| FS-1 | 150 | 150 |
| FS-2 | 200 | — |
| FS-3 | — | 150 |
| FS-4 | — | 130 |
| FS-5 | 100 | — |
| FS-6 | 100 | — |
| FS-7 | 120 | — |
| WR-1 | 150 | 150 |
| WR-2 | 130 | — |
| WR-3 | — | 200 |
| WR-4 | — | 150 |
| WR-6 | 130 | — |

### Application Example 3

### Curing of a Yellow Pigmented Composition

The procedure of example 1 is repeated, however instead of the hexafunctional polyester acrylate EBECRYL® 830 using the following polyesteracrylates and the photoinitiators indicated below:

| | | |
|---|---|---|
| F-2: | EBECRYL ® 800 | tetrafunctional polyester acrylate |
| F-3: | EBECRYL ® 810 | tetrafunctional polyester racrylate |
| F-4: | EBECRYL ® 885 | trifunctional polyester acrylate |

All EBECRYL® resins are supplied by Cytec Surface Specialties. The resins, besides the functionality, further differ in the molecular weight distribution, composition of resin, viscosity, reactivity, glass transition point. The corresponding information on the resins can be found in the information brochures and technical data sheets published by Cytec Surface Specialties (or formerly UCB Surface Specialties).

Following photoinitiators are tested:

| photoinitiator | [% by weight] |
|---|---|
| P-4 | 3.87 |
| P-6 | 2.70 |

The determination of the maximum curable wet film thickness also is performed as described in example 1. The results are listed in table 5.

TABLE 5

| | WFTₘₐₓ [μm] | WFTₘₐₓ [μm] |
|---|---|---|
| resin | P-4 | P-6 |
| F-2 | 150 | 150 |
| F-3 | 130 | 150 |
| F-4 | 80 | 80 |

1. A photocurable composition, comprising
(a) at least one ethylenically unsaturated photopolymerizable compound;
(b) at least one curing agent; and
(c) at least one colorant;
characterized in that
the ethylenically unsaturated photopolymerizable compound (a) is a polyester acrylate;
the curing agent (b) is a photoinitiator compound of the phenylglyoxylate type; and the colorant (c) is a uv- and short-vis-absorbing non-white colorant.

2. A photocurable composition according to claim 1, wherein the colorant (c) is a colored pigment or dye selected from the group consisting of yellow, orange, red, green and black pigments or dyes.

3. A photocurable composition according to claim 1,
wherein the curing agent of the phenylglyoxylate type is a compound of the formula I or Ia 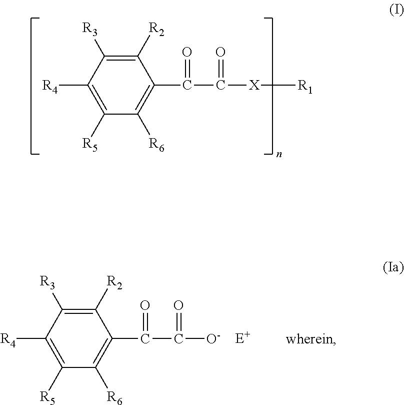
n is 1 or 2;
X is O, S or NR₁₂;
R₁, if n is 1, is hydrogen; C₁-C₂₀alkyl optionally substituted by OR₇ and/or phenyl; C₂-C₂₀alkyl interrupted by one or more O and optionally substituted by OR₇ and/or phenyl; phenyl optionally substituted by C₁-C₁₂alkyl, cyclopentyl, cyclohexyl, OR₇, SR₇ and/or NR₈R₉; C₃-C₁₂cycloalkyl or C₂-C₁₂alkenyl;
R₁, if n is 2, is C₁-C₂₀alkylene optionally substituted by OR₇ and/or phenyl; C₂-C₂₀alkylene interrupted by one or more O and optionally substituted by OR₇ and/or phenyl; phenylene optionally substituted by C₁-C₁₂alkyl, cyclopentyl, cyclohexyl, OR₇, SR₇ and/or NR₈R₉; C₃-C₁₂cycloalkylene or C₂-C₁₂alkenylene or is one of the groups 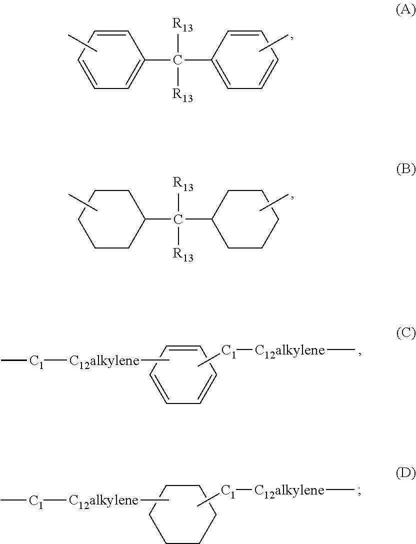
R₂, R₃, R₄, R₅, and R₆ independently of each other are hydrogen; C₁-C₂₀alkyl which optionally is substituted by phenyl, OR₇, SR₇ and/or NR₈R₉; C₂-C₂₀alkyl which is interrupted by one or more O and optionally is substituted by phenyl, OR₇, SR₇ and/or NR₈R₉; C₃-C₁₂cycloalkyl; C₂-C₁₂alkenyl; phenyl which optionally is substituted by one or more C₁-C₁₂alkyl, OR₇, SR₇ and/or NR₈R₉; or are OR₇, SR₇, or NR₈R₉;
R₇ is hydrogen, C₁-C₂₀alkyl, optionally substituted by OH, OR₁₀ and/or phenyl; C₂-C₂₀alkyl interrupted by one or more O and optionally substituted by OH, OR₁₀ and/or phenyl; phenyl, optionally substituted by one or more C₁-C₁₂alkyl;
R₈ and R₉ independently of each other are hydrogen, C₁-C₂₀alkyl, optionally substituted by OH, OR₁₀ and/or phenyl; C₂-C₂₀alkyl interrupted by one or more O and optionally substituted by OH, OR₁₀ and/or phenyl; phenyl, optionally substituted by one or more C₁-C₁₂alkyl; COR₁₁; or R₈ and R₉ together with the N-atom to which they are attached form a 5-, 6- or 7-membered ring which optionally is interrupted by O or by NR₁₂;
and wherein R₇, R₈ or R₈ as OR₇, SR₇ or NR₈R₉ with further substituents at the phenyl ring or with a C-atom of the phenyl ring form a 5- or 6-membered ring;
R₁₀ is C₁-C₂₀alkyl;
R₁₁ is C₁-C₂₀alkyl or OR₁₀;
R₁₂ is hydrogen; C₁-C₂₀alkyl, optionally substituted by phenyl, OH and/or OR₁₀; C₂-C₂₀alkyl interrupted by one or more O and optionally substituted by phenyl, OH and/or OR₁₀; C₃-C₁₂cycloalkyl; phenyl, optionally substituted by one or more C₁-C₁₂alkyl, OR₇, SR₇ and/or NR₈R₉;
R₁₃ is hydrogen or C₁-C₂₀alkyl;
E is a cation.

4. A photocurable composition according to claim 1, comprising, in addition to components (a) and (b) and (c), further photoinitiators (e) and/or further additives (d).

5. A photocurable composition according to claim 1, comprising 0.05 to 15% by weight of the curing agent (b), based on the total composition.

6. A photocurable composition according to claim 1, comprising 1 to 60% by weight of the colorant (c), based on the total composition.

7. A process for curing colored compositions, which process comprises applying a colored composition according to claim 1 on at least one surface of a substrate and irradiating the composition with light in the range from 200 to 600 nm.

8. A process according to claim 7 wherein the composition,
is applied to the substrate in a thickness of 0.1 to 300 μm;
and subsequently is irradiated with light in the wavelength range from 200 to 600 nm.

9. (canceled)

10. A coated substrate which has been coated on at least one surface with a colored composition according to claim 1.

11. Pigmented surface coatings, printing inks, screen printing inks, offset printing inks, flexographic printing inks, powder coatings, printing plates, adhesives, composite materials, gel coats, glass-fibre cable coatings, screen printing stencils, resist materials, colour filters, three-dimensional objects obtained by means of stereolithography, photographic reproductions or image recording material containing a composition according to claim 1.

12. A process according to claim 7 for the preparation of pigmented surface coatings, printing inks, screen printing inks, offset printing inks, flexographic printing inks, powder coatings, printing plates, adhesives, composite materials, gel coats, glass-fibre cable coatings, screen printing stencils, resist materials, colour filters, of three-dimensional objects by means of stereolithography, of photographic reproductions or image recording material.

13. Compounds of the formula (Ib), 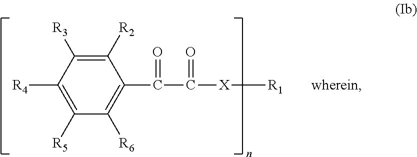
n is 1 or 2;
X is O or S;
R₁, if n is 1, is branched C₃-C₂₀alkyl or is C₅-C₈cycloalkyl, which is unsubstituted or substituted by linear or branched C₁-C₂₀alkyl;
R₁, if n is 2, is branched C₃-C₂₀alkylene;
R₂, R₃, R₄, R₅, and R₆ independently of each other are hydrogen; C₁-C₂₀alkyl which optionally is substituted by phenyl, OR₇, SR₇ and/or NR₈R₉; C₂-C₂₀alkyl which is interrupted by one or more O and optionally is substituted by phenyl, OR₇, SR₇ and/or NR₈R₉; C₃-C₁₂cycloalkyl; C₂-C₁₂alkenyl; phenyl which optionally is substituted by one or more C₁-C₁₂alkyl, OR₇, SR₇ and/or NR₈R₉; or are OR₇, SR₇, or NR₈R₉;
provided that at least one of R₂, R₃, R₄, R₅, and R₆ is SR₇;
R₇ is hydrogen, C₁-C₂₀alkyl, optionally substituted by OH, OR₁₀ and/or phenyl; C₂-C₂₀alkyl interrupted by one or more O and optionally substituted by OH, OR₁₀ and/or phenyl; phenyl, optionally substituted by one or more C₁-C₁₂alkyl;
R₈ and R₉ independently of each other are hydrogen, C₁-C₂₀alkyl, optionally substituted by OH, OR₁₀ and/or phenyl; C₂-C₂₀alkyl interrupted by one or more O and optionally substituted by OH, OR₁₀ and/or phenyl; phenyl, optionally substituted by one or more C₁-C₁₂alkyl; COR₁₁; or R₈ and R₈ together with the N-atom to which they are attached form a 5-, 6- or 7-membered ring which optionally is interrupted by O or by NR₁₂;
and wherein R₇, R₈ or R₈ as OR₇, SR₇ or NR₈R₉ with further substituents at the phenyl ring or with a C-atom of the phenyl ring form a 5- or 6-membered ring;
R₁₀ is C₁-C₂₀alkyl;
R₁₁ is C₁-C₂₀alkyl or OR₁₀; and
R₁₂ is hydrogen; C₁-C₂₀alkyl, optionally substituted by phenyl, OH and/or OR₁₀; C₂-C₂₀alkyl interrupted by one or more O and optionally substituted by phenyl, OH and/or OR₁₀; C₃-C₁₂cycloalkyl; phenyl, optionally substituted by one or more C₁-C₁₂alkyl, OR₇, SR₇ and/or NR₈R₉.

14. Compounds of the formula (Ib) according to claim 13, wherein
R₄ is SR₇;
R₂, R₃, R₅, and R₆ independently of each other are hydrogen; and
R₇ is C₁-C₂₀alkyl.

15. The compounds

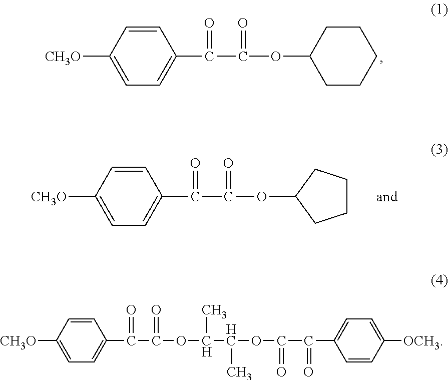

16. A photopolymerizable composition, comprising
(a) at least one ethylenically unsaturated photopolymerizable compound; and
(b) as photoinitiator at least one compound of the formula (Ib) according to claim 13.

17. A photopolymerizable composition according to claim 16 comprising in addition to the photoinitiator (b) of the formula (Ib), at least one further photoinitiator (b1) and/or further coinitiators (d) and/or other additives (e).

18. A photopolymerizable composition according to claim 16, comprising 0.05 to 25% by weight of the photoinitiator (b), or the photoinitiator (b) and at least one further photoinitiator (b1), based on the composition.

19. A process for the photopolymerization of compounds containing ethylenically unsaturated double bonds, which comprises irradiating a composition according to claim 16 with electromagnetic radiation in the range from 150 to 600 nm, or with electron beam or with X-rays.

20-21. (canceled)

22. A process according to claim 19 for producing pigmented and nonpigmented paints and varnishes, powder coatings, printing inks, printing plates, adhesives, pressure sensitive adhesives, dental compositions, gel coats, photoresists for electronics, electroplating resists, etch resists, both liquid and dry films, solder resists, resists to manufacture color filters for a variety of display applications, resists to generate structures in the manufacturing processes of plasma-display panels, electroluminescence displays and LCD, spacers for LCD, for holographic data storage (HDS), as composition for encapsulating electrical and electronic components, for producing magnetic recording materials, micromechanical parts, waveguides, optical switches, plating masks, etch masks, colour proofing systems, glass fibre cable coatings, screen printing stencils, for producing three-dimensional objects by means of stereolithography, as image recording material, for holographic recordings, microelectronic circuits, decolorizing materials, decolorizing materials for image recording materials, for image recording materials using microcapsules, as a photoresist material for a UV and visible laser direct imaging system, as a photoresist material used for forming dielectric layers in a sequential build-up layer of a printed circuit board.

23. Coated substrate which is coated on at least one surface with a composition according to claim 16.

24. A photopolymerizable composition, comprising
(a) at least one ethylenically unsaturated photopolymerizable compound; and
(b) as photoinitiator at least one compound according to claim 15.

25. A process for the photopolymerization of compounds containing ethylenically unsaturated double bonds, which comprises irradiating a composition according to claim 24 with electromagnetic radiation in the range from 150 to 600 nm, or with electron beam or with X-rays.
